(19) United States
(12) Patent Application Publication
(10) Pub. No.: US 2025/0326767 A1
(43) Pub. Date: Oct. 23, 2025

(54) NOVEL BENZOTHIAZOLE DERIVATIVES

(71) Applicants: CARNA BIOSCIENCES, INC., Hyogo (JP); Sumitomo Pharma Co., Ltd., Osaka (JP)

(72) Inventors: Yuko ASAMITSU, Kobe-shi, Hyogo (JP); Wataru KAWAHATA, Kobe-shi, Hyogo (JP); Hiroe NAKAYAMA, Kobe-shi, Hyogo (JP); Masaaki SAWA, Kobe-shi, Hyogo (JP)

(73) Assignees: CARNA BIOSCIENCES, INC., Hyogo (JP); Sumitomo Pharma Co., Ltd., Osaka (JP)

(21) Appl. No.: 18/292,080

(22) PCT Filed: Jul. 27, 2022
(86) PCT No.: PCT/JP2022/028904

§ 371 Date: May 9, 2024

(30) Foreign Application Priority Data
Jul. 28, 2021 (JP) 2021-123152

(51) Int. Cl. C07D 513/04; A61K 31/429; A61K 31/513; A61K 45/06; C07D 519/00

(52) U.S. Cl. CPC C07D 513/04; A61K 31/429; A61K 31/513; A61K 45/06; C07D 519/00

(57) ABSTRACT

The present invention provides a benzothiazole derivative represented by formula (I):

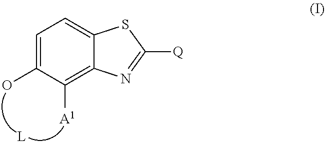

(in the formula, A¹, L and Q are as defined in the description)
or a pharmaceutically acceptable salt thereof, which exhibit a DYRK inhibitory activity and is useful as a pharmaceutical.

### TECHNICAL FIELD

The present invention relates to a medicament, particularly a novel benzothiazole derivative having a DYRK inhibitory effect or a pharmaceutically acceptable salt thereof.

### BACKGROUND ART

DYRK (dual-specificity tyrosine-phosphorylation regulated kinase) is one of the bispecific protein kinases that phosphorylate tyrosine, serine, and threonine. DYRK functions as a tyrosine kinase only in the case of autophosphorylation and catalyzes the phosphorylation of serine or threonine residues on exogenous substrates. Five members of the DYRK family are known in humans: DYRK1A, DYRK1B, DYRK2, DYRK3, and DYRK4 (Non Patent Literature 1).

It has been widely reported that DYRK1A is associated with neuropsychiatric diseases. For example, in patients with Alzheimer's disease, the expression of β-amyloid is significantly consistent with that of DYRK1A (Non Patent Literature 2), and it is speculated that DYRK1A is involved in abnormal phosphorylation of a tau protein (Tau), which is considered to contribute to the onset of Alzheimer's disease (Non Patent Literature 3).

In addition, Parkinson's disease is a neurodegenerative disease caused by the degeneration of dopamine neurons, which are important for motor function, but one of the causes is considered to be mitochondrial dysfunction (Non Patent Literature 4). An enzyme involved in protein degradation called Parkin is known to metabolize abnormal mitochondria and suppress abnormal accumulation, but DYRK1A has been reported to suppress the activity of this parkin protein (Non Patent Literature 5).

The gene for DYRK1A is located in the Down's syndrome critical region, and it has been reported that mice overexpressing DYRK1A exhibit neuropsychiatric dysfunction and appear like Down's syndrome (Non Patent Literature 6). It has also been reported that DYRK1A expression is increased in the brain of patients with Down's syndrome and Down's syndrome-like model mice (Non Patent Literature 7). These reports suggest that DYRK1A is involved in the onset of neurological symptoms in the patients with Down's syndrome (Non Patent Literature 8).

In addition, it has been reported that early-onset Alzheimer's disease occurs frequently in patients with Down's syndrome, thus indicating that DYRK1A is closely related to Alzheimer's disease (Non Patent Literature 8).

Therefore, compounds inhibiting DYRK1A are considered useful for treating neuropsychiatric diseases such as Alzheimer's disease, Down's syndrome, mental retardation, memory impairment, memory loss, and Parkinson's disease.

Recently, it has been reported that DYRK1A is highly expressed in brain tumors such as glioblastoma and regulates the expression of an epidermal growth factor receptor (EGFR) (Non Patent Literature 9). Therefore, compounds inhibiting DYRK1A are considered useful for treating EGFR-dependent cancers by suppressing the proliferation of cancer cells in EGFR-dependent brain tumors and other tumors.

Compounds inhibiting the family enzymes DYRK1B, DYRK2, and DYRK3 are also considered to have various pharmaceutical applications. For example, it has been reported that DYRK1B is highly expressed in quiescent (GO-phase) cancer cells and contributes to resistance to various chemotherapeutic agents (Non Patent Literature 10). It has also been reported that inhibition of DYRK1B promotes withdrawal from the GO phase and enhances sensitivity to chemotherapeutic agents (Non Patent Literature 11). Therefore, compounds inhibiting DYRK1B are considered useful for treating pancreatic cancer, ovarian cancer, osteosarcoma, colorectal cancer, and lung cancer (Non Patent Literatures 11, 12, 13, 14, and 15).

It is suggested that DYRK2 controls p53 to induce apoptosis in response to DNA damages (Non Patent Literature 16). Furthermore, it has been reported that compounds inhibiting DYRK3 are useful for treating sickle cell anemia and chronic kidney disease (Non Patent Literature 17).

In addition to Patent Literature 1 for compounds inhibiting DYRK, Patent Literature 2 has been reported for DYRK1A and DYRK1B inhibitors. However, the benzothiazole derivative of the present invention is not disclosed therein.

### PRIOR ART LITERATURE(S)

### Patent Literature(s)

Patent Literature 1: WO2010/10797
Patent Literature 2: WO2013/26806

### Non-Patent Literature(s)

Non-Patent Literature 1: Becker W. et al., J. Biol. Chem., 1998, 273, 25893-25902
Non-Patent Literature 2: Kimura R. et al., Hum. Mol. Genet., 2007, 16, 15-23
Non-Patent Literature 3: Ryoo S R. et al., J. Biol. Chem., 2007, 282, 34850-34857
Non-Patent Literature 4: Narendra D. et al., J. Cell. Biol., 2008, 183, 795-803
Non-Patent Literature 5: Im E., J. Neurochem., 2015, 134, 756-768
Non-Patent Literature 6: Branchi I. et al., J. Neuropathol. Exp. Neurol., 2004, 63, 429-440
Non-Patent Literature 7: Dowjat W K. et al., Neurosci. Lett., 2007, 413, 77-81
Non-Patent Literature 8: Wegiel J. et al., FEBS J., 2011, 278, 236-245
Non-Patent Literature 9: Pozo N. et al., J. Clin. Invest., 2013, 123, 2475-2487.
Non-Patent Literature 10: Deng X. et al., Cancer Res., 2006, 66, 4149-4158.
Non-Patent Literature 11: Ewton D Z. et al., Mol. Cancer Ther., 2011, 10, 2104-2114.
Non-Patent Literature 12: Deng X. et al., Genes Cancer., 2014, 5, 201-211.
Non-Patent Literature 13: Yang C. et al., Carcinogenesis., 2010, 31, 552-558.
Non-Patent Literature 14: Jin K. et al., J. Biol. Chem., 2009, 284, 22916-22925.
Non-Patent Literature 15: Gao J et al., Cancer Cell Int. 2013, 13, 2
Non-Patent Literature 16: Taira N. et al., Mol. Cell., 2007, 25, 725-738.
Non-Patent Literature 17: Bogacheva O. et al., J. Biol. Chem., 2008, 283, 36665-36675.

### SUMMARY OF THE INVENTION

### Problem to be Solved by the Invention

An object of the present invention is to provide a novel compound that has a DYRK inhibitory effect and is useful as a medicament.

### Means for Solving the Problem

The object of the present invention is achieved by the following (1) to (17).

(1) A benzothiazole derivative of the following formula (I):

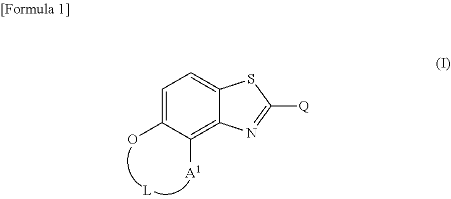

wherein A¹ represents an oxygen atom or optionally substituted methylene; L represents optionally substituted methylene or optionally substituted ethylene; and
Q represents the following structure (a) or (b):

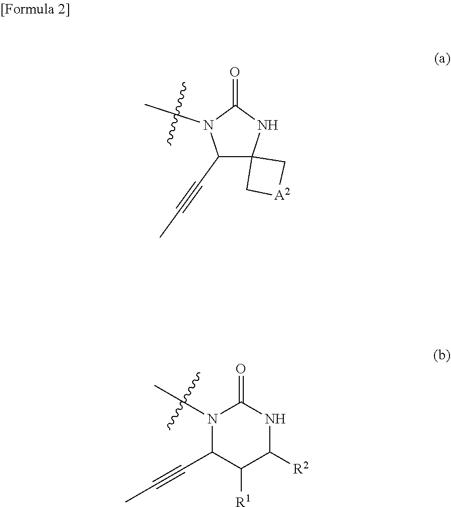

wherein A² represents, independently of A¹, a bond, optionally substituted methylene, optionally substituted ethylene, or an oxygen atom; and
R¹ and R² represent each independently a hydrogen atom or an optionally substituted lower alkyl group, or a pharmaceutically acceptable salt thereof.
   (2) The benzothiazole derivative according to (1) above or a pharmaceutically acceptable salt thereof, wherein in formula (I) above, A¹ and L are methylene, and Q is structure (a) or (b).
   (3) The benzothiazole derivative according to (1) above or a pharmaceutically acceptable salt thereof, wherein in formula (I) above, A¹ is an oxygen atom, L is ethylene, and Q is structure (a) or (b).
   (4) The benzothiazole derivative according to (2) above or a pharmaceutically acceptable salt thereof, wherein Q is structure (a) in formula (I) above.
   (5) The benzothiazole derivative according to (2) above or a pharmaceutically acceptable salt thereof, wherein Q is structure (b) in formula (I) above.
   (6) The benzothiazole derivative according to (3) above or a pharmaceutically acceptable salt thereof, wherein Q is structure (a) in formula (I) above.
   (7) The benzothiazole derivative according to (3) above or a pharmaceutically acceptable salt thereof, wherein Q is structure (b) in formula (I) above.
   (8) A benzothiazole derivative according to Examples 1 to 23 below or a pharmaceutically acceptable salt thereof.
   (9) A medicament comprising the benzothiazole derivative according to any one of (1) to (8) above or a pharmaceutically acceptable salt thereof as an active ingredient.
   (10) The pharmaceutical composition comprising the benzothiazole derivative according to any one of (1) to (8) above or a pharmaceutically acceptable salt thereof as an active ingredient.
   (11) A therapeutic agent and/or a prophylactic agent for a disease involving DYRK, comprising the benzothiazole derivative according to any one of (1) to (8) above or a pharmaceutically acceptable salt thereof as an active ingredient.
   (12) The therapeutic agent and/or the prophylactic agent according to (11) above, wherein the disease involving DYRK is frontotemporal dementia, progressive supranuclear palsy, corticobasal degeneration, Lewy body dementia, vascular dementia, a traumatic brain injury, chronic traumatic encephalopathy, stroke, Alzheimer's disease, Parkinson's disease, Down's syndrome, depression and mental retardation associated therewith, memory impairment, memory loss, learning disability, intellectual disability, cognitive impairment, mild cognitive impairment, dementia symptoms or brain tumor, pancreatic cancer, ovarian cancer, osteosarcoma, colorectal cancer, lung cancer, bone resorption disease, osteoporosis, sickle cell anemia, chronic kidney disease or bone resorption disease.
   (13) A method for treating and/or preventing a disease involving DYRK, comprising administering to a patient in need of the treatment a therapeutically effective amount of a compound according to any one of (1) to (8) above or a pharmaceutically acceptable salt thereof.
   (14) Use of the benzothiazole derivative according to any one of (1) to (8) above or a pharmaceutically acceptable salt thereof, for producing a therapeutic agent and/or a prophylactic agent for a disease involving DYRK.
   (15) The benzothiazole derivative according to any one of (1) to (8) above or a pharmaceutically acceptable salt thereof, for use in the treatment and/or prevention of a disease involving DYRK.
   (16) A medicament comprising the medicament according to (9) above in combination with at least one or more drugs selected from drugs classified as anticancer agents, antipsychotic drugs, antidementia drugs, antiepileptic drugs, antidepressants, gastrointestinal agents, thyroid hormone preparations, or antithyroid drugs.
   (17) The medicament according to (16) above, for treating frontotemporal dementia, progressive supranuclear palsy, corticobasal degeneration, Lewy body dementia, vascular dementia, a traumatic brain injury, chronic traumatic encephalopathy, stroke, Alzheimer's disease, Parkinson's disease, Down's syndrome, depression and a complication associated therewith, mental retardation, memory impairment, memory loss, learning disability, intellectual disability, cognitive impairment, mild cognitive impairment, dementia symptoms or brain tumor, pancreatic cancer, ovarian cancer, osteosarcoma, colorectal cancer, lung cancer, bone resorption disease, osteoporosis, sickle cell anemia, chronic kidney disease or bone resorption disease in combination with at least one or more drugs selected from drugs classified as anticancer agents, antipsychotic drugs, antidementia drugs, antiepileptic drugs, antidepressants, gastrointestinal agents, thyroid hormone preparations, or antithyroid drugs.

### Effect of the Invention

The present inventors have intensively studied so as to achieve the above object and found that a novel benzothiazole derivative of formula (I) and a pharmaceutically acceptable salt thereof have an excellent DYRK inhibitory effect, and thus completed the present invention. Compounds provided by the present invention are useful as a therapeutic agent for diseases which are known to be involved in abnormal cell response through DYRK1A, for example, Alzheimer's disease, Parkinson's disease, Down's syndrome, neuropsychiatric disorder such as depression, mental retardation associated therewith, memory impairment, memory loss, learning disability, intellectual disability, cognitive impairment, mild cognitive impairment, progression of dementia symptoms or a prophylactic agent for dementia onset; and a prophylactic or therapeutic pharmaceutical (pharmaceutical composition) for tumors such as brain tumors. The compounds provided by the present invention are DYRKIB inhibitors that are useful as a prophylactic or therapeutic pharmaceutical (pharmaceutical composition) for tumors such as pancreatic cancer, ovarian cancer, osteosarcoma, colorectal cancer, and lung cancer. Since DYRK2 controls p53 to induce apoptosis in response to DNA damages, the compounds provided by the present invention are further useful as a prophylactic or therapeutic pharmaceutical (pharmaceutical composition) for bone resorption disease and osteoporosis. The compounds provided by the present invention are also DYRK3 inhibitors that are useful as a prophylactic or therapeutic pharmaceutical (pharmaceutical composition) for sickle cell anemia, chronic kidney disease, bone resorption disease, and osteoporosis. The compounds are also useful, as a compound inhibiting DYRK, for reagents to be used in pathological imaging and for reagents for basic experiments and research regarding the above diseases.

### BEST MODE TO CARRY OUT THE INVENTION

The present invention will be described in detail below.

The novel benzothiazole derivative of the present invention is a compound of the following formula (I):

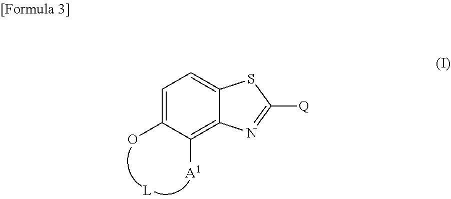

wherein A¹ represents an oxygen atom or optionally substituted methylene; L represents optionally substituted methylene or optionally substituted ethylene; and
Q represents the following structure (a) or (b):

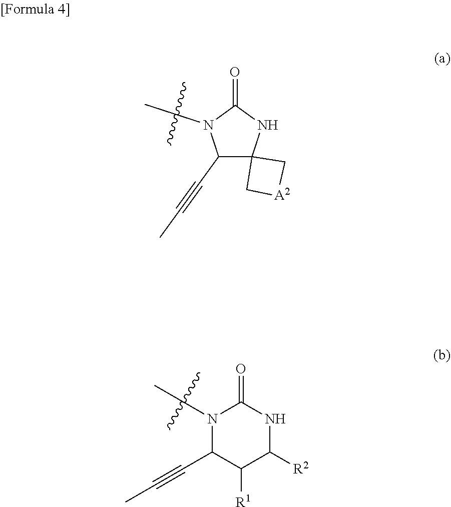

wherein A² represents, independently of A¹, a bond, optionally substituted methylene, optionally substituted ethylene, or an oxygen atom; and
R¹ and R² represent each independently a hydrogen atom or an optionally substituted lower alkyl group.

The term “DYRK” represents a dual-specificity tyrosine-phosphorylation regulated protein kinase and means one or more DYRK family members (DYRK1A, DYRK1B, DYRK2, DYRK3, and DYRK4).

The term “lower alkyl group” means a linear or branched saturated hydrocarbon group of 1 to 6 carbon atoms (C₁₋₆ alkyl group). The lower alkyl group preferably includes a “C₁₋₄ alkyl group” and more preferably a “C₁₋₃ alkyl group”. Specific examples of the “lower alkyl group” include a methyl group, an ethyl group, an n-propyl group, a 1-methylethyl group, an n-butyl group, a tert-butyl group, a 1-methylpropyl group, a 2-methylpropyl group, an n-pentyl group, a 1,1-dimethylpropyl group, a 1,2-dimethylpropyl group, a 1-methylbutyl group, a 2-methylbutyl group, a 4-methylpentyl group, a 3-methylpentyl group, a 2-methylpentyl group, a 1-methylpentyl group, and a hexyl group.

For a “substituent” of the optionally substituted lower alkyl group, the group may have one or more of any type of substituents at any chemically allowable position, and when the above group has two or more substituents, the substituents may be the same or different, unless otherwise specified. The substituent specifically includes, for example, a C₃₋₆ cycloalkyl group, a halogen atom, a C₁₋₄ alkoxy group, a cyano group, a benzyloxy group, a phenyl group, a hydroxy group, a methanesulfonyl group, and a substituted or unsubstituted amino group.

The term “C₃₋₆ cycloalkyl group” means a cyclic saturated hydrocarbon group of 3 to 6 carbon atoms and also includes those having a partially unsaturated bond and a cross-linked structure. Specific examples of the “C₃₋₆ cycloalkyl group” include a cyclopropyl group, a cyclobutyl group, a cyclopentyl group, and a cyclohexyl group.

A “halogen atom” refers to a chlorine atom (Cl), a bromine atom (Br), a fluoride atom (F), and iodine atom (I), and in particular, is preferably Cl, Br, and F.

The term “C₁₋₄ alkoxy group” means an oxy group substituted with an alkyl group of the “C₁₋₄ alkyl group”. The “C₁₋₄ alkoxy group” preferably includes a “C₁₋₃ alkoxy group”. Specific examples of the “C₁₋₄ alkoxy group” include a methoxy group, an ethoxy group, a propoxy group, a 1-methylethoxy group, a butoxy group, a 1,1-dimethylethoxy group, a 1-methylpropoxy group, and a 2-methylpropoxy group.

A “substituent” of the optionally substituted methylene or the optionally substituted ethylene in A¹, A², and L each independently include a halogen atom and a lower alkyl group.

Examples of the pharmaceutically acceptable salt of compound (I) of the present invention include inorganic acid salts with hydrochloric acid, sulfuric acid, carbonic acid, and phosphoric acid; and organic acid salts with fumaric acid, maleic acid, methanesulfonic acid, and p-toluenesulfonic acid. The present invention also encompasses ammonium salts, in addition to salts with an alkali metal such as sodium and potassium; with an alkaline earth metal such as magnesium and calcium; with an organic amine such as lower alkylamine and lower alcoholamine; and with a basic amino acid such as lysine, arginine, and ornithine.

Isomers may exist in compound (I) of the present invention, for example, depending on the type of the substituent. The isomers may be herein described by a chemical structure of only one form thereof, but the present invention encompasses all isomers (geometrical isomer, stereoisomer, tautomer, etc.) which can be structurally formed, and also encompasses isomers alone, or a mixture thereof.

Compound (I) or a pharmaceutically acceptable salt thereof of the present invention can be produced, for example, by the methods described below. When a defined group is chemically affected under the conditions of an exemplified method in the production method shown below or is unsuitable for use to carry out the method, it is possible to easily produce them by a method which is usually used in organic synthetic chemistry, for example, a method of applying means such as protection or deprotection of a functional group [T. W. Greene, Protective Groups in Organic Synthesis 3rd Edition, John Wiley & Sons, Inc., 1999]. If necessary, the order of reaction steps such as introduction of substituents cab also be changed.

Meanings of abbreviations and symbols used in the following description are as follows.
DCM: dichloromethane
THF: tetrahydrofuran
DMF: N, N-dimethyl formamide
TEA: triethylamine
DMA: N, N-dimethyl acetamide
DMSO: dimethyl sulfoxide

### [Preparative Method of Compound (I) of the Present Invention]

Compound (I-a) of the present invention, in which Q is represented by structure (a) in the formula (I), can be produced, for example, according to Scheme 1:

scheme 1

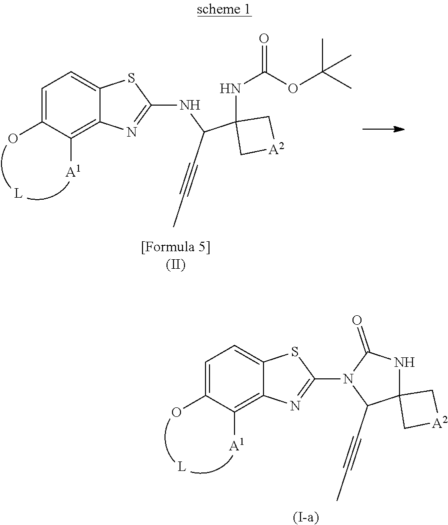

wherein A¹, A², and L are the same as defined in the above.

Compound (I-a) of the present invention can be produced by intramolecular cyclization of compound (II). In other words, compound (I-a) can be obtained by treating compound (II) with 1 to 10 molar equivalents of a base such as potassium carbonate in a solvent such as DMF. Any solvent may be used as long as it is inert to the reaction. Although it is not particularly limited, for example, DMF, DMA, and THE can be used, and DMF can be preferably used. The reaction can be carried out within several minutes to several days at a temperature ranging from 0° C. to 150° C., and it is possible to carry out the reaction preferably for 1 hour to 24 hours at 50° C. to 130° C.

Compound (I-a) can also be produced by reacting an amine obtained by deprotecting a tert-butoxycarbonyl group of compound (II) with a condensing reagent such as 1,1′-carbonyldiimidazole (CDI) and di(N-succinimidyl) carbonate (DSC) in a solvent.

Compound (II) to be used as a raw material of Scheme 1 can be produced, for example, by the method shown in Scheme 2:

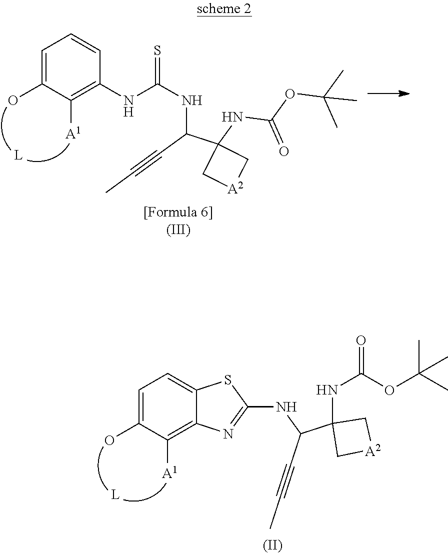

wherein A¹, A², and L are the same as defined in the above.

Compound (II) can be produced by reacting compound (III) with a brominating agent to form a thiazole ring. In other words, compound (II) can be obtained by reacting compound (III) with a large excess of acetic acid and 0.5 to 1.5 molar equivalents of bromine in solvents such as acetonitrile and DCM. Compound (II) can also be synthesized by reacting compound (III) with 1 to 20 molar equivalents of sodium hydrogencarbonate and a brominating agent such as 0.5 to 1.5 molar equivalents of benzyltrimethylammonium tribromide. Any solvent may be used as long as it is inert to the reaction. Although it is not particularly limited, for example, acetonitrile, DCM, and chloroform can be used. The reaction can be carried out within several minutes to several days at a temperature ranging from −20° C. to 50° C., and it is possible to synthesize the product preferably by reacting from several minutes to 24 hours at 0° C. to room temperature.

Compound (III) to be used as a raw material of Scheme 2 can be produced, for example, by the method shown in Scheme 3:

scheme 3

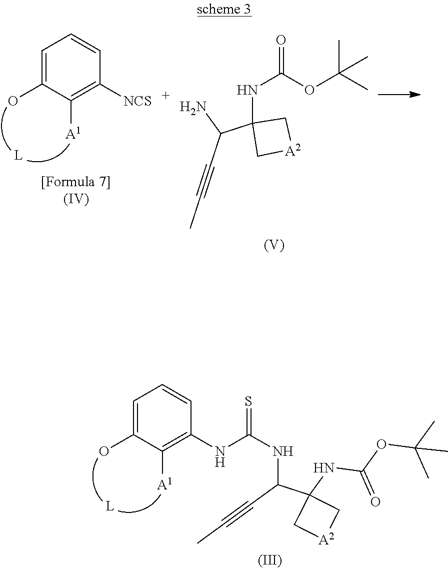

wherein A¹, A², and L are the same as defined in the above.

Compound (III) can be produced by reacting isothiocyanate (IV) with amine (V). In other word, compound (III) can be obtained by reacting the isothiocyanate (IV) with the amine (V) in a solvent, if necessary, in the presence of a base. Any solvent may be used as long as it is inert to the reaction. Although it is not particularly limited, for example, THE and ethanol can be used, and as the base, 0.5 to 1.5 molar equivalents of a base such as TEA or sodium ethoxide can be used. The reaction can be carried out within several minutes to several days at a temperature ranging from 0 to 100° C., and it is possible to carry out the reaction preferably for 1 hour to 24 hours at room temperature to 40° C.

Compound (I-b) of the present invention, in which Q is represented by structure (b) in the formula (I), can be produced, for example, according to Scheme 4:

scheme 4

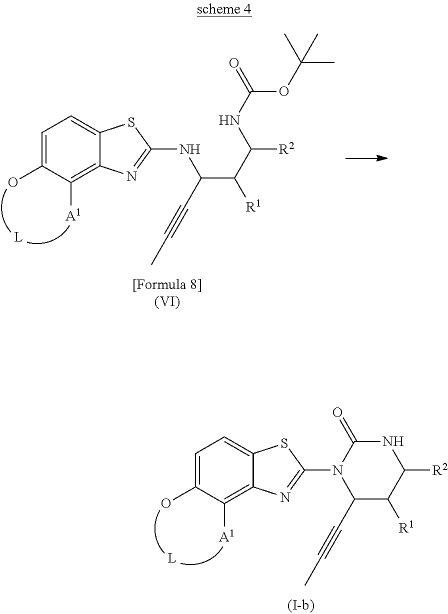

wherein A¹, R¹, R², and L are the same as defined in the above.

Compound (I-b) of the present invention can be produced by intramolecular cyclization of compound (VI). In other words, compound (I-b) can be obtained by reacting compound (VI) under the same conditions as in Scheme 1.

Compound (VI) to be used as a raw material of Scheme 4 can be produced, for example, by the method shown in Scheme 5:

scheme 5

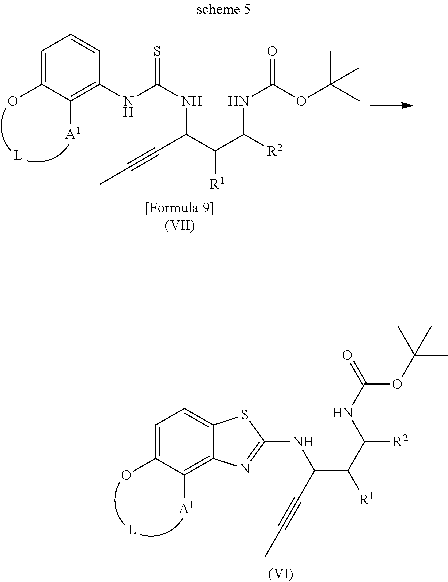

wherein A¹, R¹, R², and L are the same as defined in the above.

Compound (VI) can be produced by reacting compound (VII) with a brominating agent to form a thiazole ring. In other words, compound (VI) can be obtained by reacting compound (VII) under the same conditions as in Scheme 2.

Compound (VII) to be used as a raw material of Scheme 5 can be produced, for example, by the method shown in Scheme 6:

scheme 6

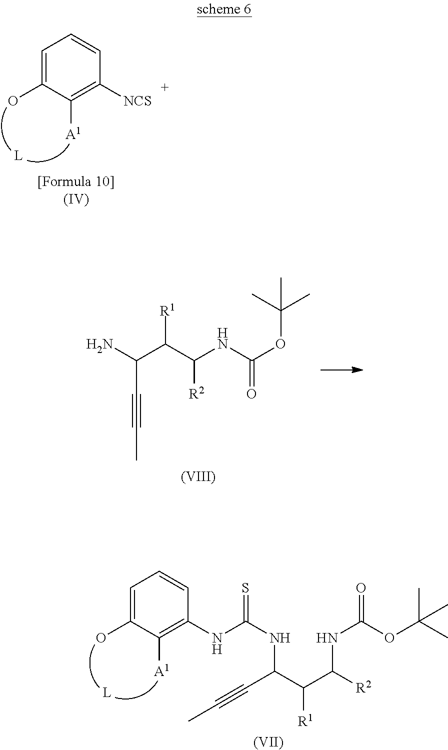

wherein A¹, R¹, R², and L are the same as defined in the above.

Compound (VII) can be produced by reacting isothiocyanate (IV) with amine (VIII). In other words, compound (VII) can be obtained by reacting the isothiocyanate (IV) with the amine (VIII) under the same conditions as in Scheme 3.

Isothiocyanate (IV) to be used as a raw material of Schemes 3 and 6 can be produced, for example, by the method shown in Scheme 7:

scheme 7

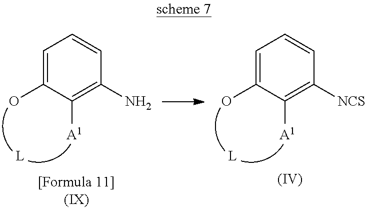

wherein A¹ and L are the same as defined in the above.

The isothiocyanate (IV) can be obtained by reacting amine (IX) with 1 to 10 molar equivalents of thiophosgene in an aqueous solution. The reaction can be carried out within several minutes to 24 hours at a temperature ranging from −30° C. to room temperature, and it is possible to carry out the reaction preferably for 1 hour to 4 hours at −10° C. to 0° C.

Amine (IX) to be used as a raw material of Scheme 7 can be obtained as a commercially available product or produced by a known method or a method which is usually used in organic synthetic chemistry.

Compound (V) to be used as another raw material of Scheme 3 can be produced, for example, by the method shown in Scheme 8:

scheme 8

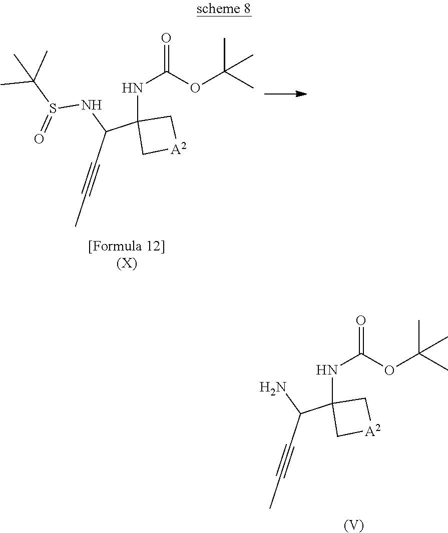

wherein A² is the same as defined in the above.

The amine (V) can be produced by deprotecting compound (X) with an acid in a solvent. Any solvent may be used as long as it is inert to the reaction. Although it is not particularly limited, for example, 1,4-dioxane can be used, and as the acid, for example, a hydrogen chloride solution such as a 0.5 to 1.5 molar equivalents of hydrogen chloride/1, 4-dioxane solution can be used. The reaction can be carried out within several minutes to several days at a temperature ranging from 0° C. to 100° C., and it is possible to carry out the reaction preferably for 1 hour to 24 hours at 0° C. to 40° C.

Compound (X) to be used as a raw material of Scheme 8 can be produced, for example, by the method shown in Scheme 9:

scheme 9

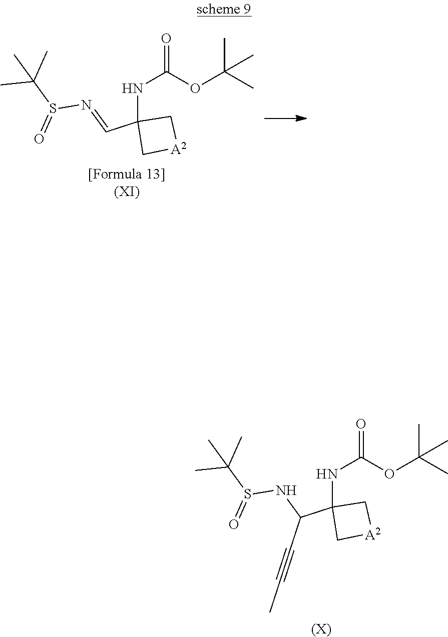

wherein A² is the same as defined in the above.

Compound (X) can be produced by the Grignard reaction of imine (XI) with 1 to 10 molar equivalents of 1-propynylmagnesium bromide in a solvent. Any solvent may be used as long as it is inert to the reaction. Although it is not particularly limited, for example, an ether solvent such as THE and 1,2-dimethoxyethane can be used. The reaction can be carried out within several minutes to 24 hours at a temperature ranging from −80° C. to room temperature, and it is possible to synthesize the product preferably by reacting for 30 minutes to 6 hours at −80° C. to −20° C. In this reaction, compound (X) can be synthesized with high stereoselectivity by using the imine moiety as an optically active asymmetric auxiliary group.

Compound (XI) to be used as a raw material of Scheme 9 can be produced, for example, by the method shown in Scheme 10:

scheme 10

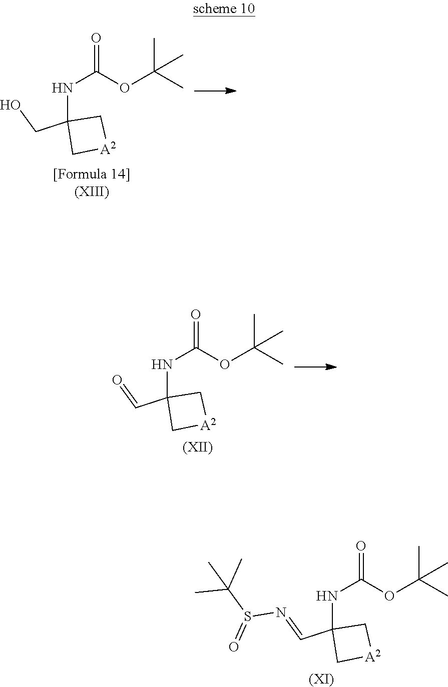

wherein A² is the same as defined in the above.

Imine (XI) can be produced by converting alcohol (XIII) into aldehyde (XII), followed by a condensation reaction with tert-butanesulfinamide. In other words, the aldehyde (XII) can be synthesized by a method which is usually used in organic synthetic chemistry, for example, a method of oxidizing a hydroxy group of the alcohol (XIII) to an aldehyde group using an oxidizing agent such as a Dess-Martin reagent. The imine (XI) can be obtained by reacting the resulting aldehyde (XII) with 1 to 5 molar equivalents of tert-butanesulfinamide in the presence of a Lewis acid or the like in a solvent. Any solvent may be used as long as it is inert to the reaction. Although it is not particularly limited, for example, a solvent such as THE and DCM can be used. The reaction can be carried out within several minutes to several days at a temperature ranging from −20° C. to 100° C., and it is possible to synthesize the product preferably by reacting from several minutes to 24 hours at room temperature to 60° C. By using optically active tert-butanesulfinamide in this reaction, the tert-butanesulfinamide can be used as an asymmetric auxiliary group in the reaction of Scheme 9.

Compound (VIII) to be used as a raw material of Scheme 6 can be produced in the same manner by the methods shown in Schemes 8 to 10 by using corresponding alcohol derivatives instead of alcohol (XIII).

It is possible to obtain compound (I) having the desired functional group at the desired position of the present invention by appropriately using the above methods in combination, and then carrying out a method usually used in organic synthetic chemistry (for example, an alkylation reaction, an acylation reaction, a carbamoylation reaction, and a carbamatation reaction of an amino group; alkoxylation, acylation, and carbamatation reactions of a hydroxyl group; or a reaction of inversely converting the group).

### [Use of Compound (I) of the Present Invention]

The compound represented by formula (I) or a pharmaceutically acceptable salt thereof of the present invention can be prepared in the form of a conventional pharmaceutical formulation (pharmaceutical composition), which is suitable for oral administration, parenteral administration, or local administration.

Formulations for oral administration include solid formulations such as tablets, granules, powders, and capsules; and liquid formulations such as syrups. These formulations can be prepared by a conventional method. The solid formulations can be prepared by using conventional pharmaceutical carriers, for example, lactose; starches such as corn starch; crystalline celluloses such as microcrystalline cellulose; and hydroxypropyl cellulose, calcium carboxymethyl cellulose, talc, and magnesium stearate. Capsules can be prepared by encapsulating thus prepared granules or powders. Syrups can be prepared by dissolving or suspending the compound represented by formula (I) or a pharmaceutically acceptable salt thereof of the present invention in an aqueous solution containing sucrose, carboxymethyl cellulose and the like.

Formulations for parenteral administration include injections such as formulations for drip infusion. Injection formulations can also be prepared by a conventional method and can be appropriately incorporated into isotonic agents (for example, mannitol, sodium chloride, glucose, sorbitol, glycerol, xylitol, fructose, maltose, and mannose), stabilizers (for example, sodium sulfite and albumin), and antiseptics (for example, benzyl alcohol and methyl p-oxybenzoate).

The dosage of the compound represented by formula (I) or a pharmaceutically acceptable salt thereof of the present invention can vary depending on types and severity of disease; age, sex, and body weight of the patient; and dosage form, and is usually within a range from 1 mg to 1,000 mg per day for adults. The compound or a pharmaceutically acceptable salt thereof can be administered once a day, or dividedly administered twice or three times a day through an oral or parenteral route.

The compound represented by formula (I) or a pharmaceutically acceptable salt thereof of the present invention can also be used, as a DYRK inhibitor, for reagents to be used in pathological imaging and for reagents for basic experiments and research regarding the above diseases.

### EXAMPLES

The present invention will be more specifically described below by way of Examples and Test Examples, but the present invention is not limited to these Examples.

Identification of the compound was carried out by hydrogen nuclear magnetic resonance spectrum (¹H-NMR) and mass spectrum (MS). ¹H-NMR is measured at 400 MHZ unless otherwise specified, and exchangeable hydrogen may not be clearly observed depending on the compound and measurement conditions. The sign “br” means a broad signal (broad). HPLC preparative chromatography was carried out by a commercially available ODS column in a gradient mode using water/methanol or water/acetonitrile (containing formic acid) as eluents, unless otherwise specified.

When the bond of the substituent at an optically active center of a compound is indicated by a wavy line, it indicates that the compound is a mixture of R and S bodies with respect to the stereochemistry at a substitution position thereof; and when the bond of the substituent at an optically active center is indicated by a solid line, it indicates that the compound is an R or S form for a substitution position thereof. Each enantiomer was obtained appropriately as a single compound by optical resolution.

### Reference Example 1

### Production of 4-isothiocyanato-2, 3-dihydrobenzofuran

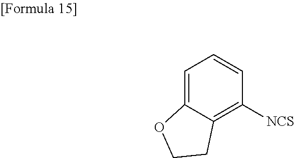

To a solution of 2,3-dihydrobenzofuran-4-amine (1 g, 7.41 mmol) in aqueous solution (10 mL), thiophosgene (1.41 mL, 18.52 mmol) was added at 0° C., and the mixture was stirred for 4 hours at room temperature. After completion of the reaction, the reaction mixture was diluted with water and extracted with ethyl acetate. The organic layer was washed with water and saturated brine solution and then dried over anhydrous sodium sulfate. After the solvent was concentrated under reduced pressure, the residue was purified by column chromatography (silica gel, petroleum ether) to afford the title compound (yield: 0.9 g).

¹H-NMR (CDCl₃) δ (ppm) 7.07 (t, J=8.0 Hz, 1H), 6.70 (dd, J=1.1, 7.9 Hz, 2H), 4.62 (t, J=8.8 Hz, 2H), 3.30 (t, J=8.6 Hz, 2H). GC-MS: 177.1 (M⁺).

### Reference Example 2

### Production of tert-butyl (RS)-(3-aminohex-4-yn-1-yl)carbamate

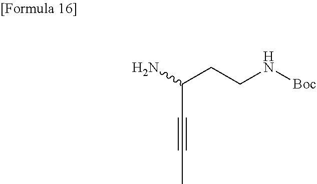

### First Step

### Production of tert-butyl (RS)-(3-((tert-butylsulfinyl)imino)propyl)carbamate

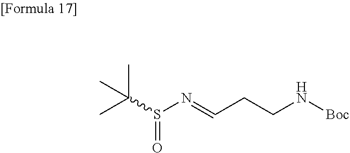 [SMILES: CC(C)(C)OC(=O)NCC/C=N/S(=O)C(C)(C)C]

To a solution of tert-butyl (3-oxopropyl)carbamate (2.57 g, 14.84 mmol) and 2-methyl-2-propanesulfinamide (2.7 g, 22.26 mmol) in THE (47.4 mL), tetraethoxytitanium (6.22 mL, 29.7 mmol) was added under a nitrogen flow and stirred overnight at room temperature. After saturated brine was added to the reaction mixture, the precipitated solid was collected by filtration, and the filtrate was extracted with ethyl acetate. The organic layer was washed with saturated brine and dried over anhydrous sodium sulfate. The residue obtained by removing the solvent was purified by column chromatography (silica gel, hexane/ethyl acetate) to afford the title compound (1.43 g).

¹H-NMR (DMSO-d₆) δ (ppm) 7.92 (t, J=4.5 Hz, 1H), 6.90 (t, J=5.8 Hz, 1H), 3.29-3.15 (m, 2H), 2.72-2.55 (m, 2H), 1.36 (s, 9H), 1.10 (s, 9H).

### Second Step

### Production of tert-butyl ((RS)-3-((RS)-tert-butylsulfinyl)amino)hex-4-yn-1-yl)carbamate

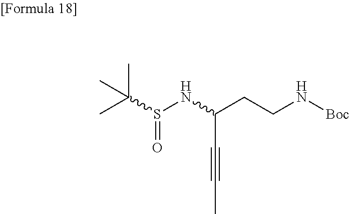 [SMILES: CC#CC(CCNC(=O)OC(C)(C)C)NS(=O)C(C)(C)C]

To a solution of tert-butyl (RS)-(3-((tert-butylsulfinyl)imino)propyl)carbamate (1.3 g, 4.7 mmol) in THF (23.52 mL), 1-propynylmagnesium bromide in 0.5 M-THF solution (18.81 mL, 9.41 mmol) was added dropwise at −20° C., stirred for 1 hour at the same temperature, and then stirred for 50 minutes at room temperature. Another 1-propynylmagnesium bromide in 0.5 M-THF solution (18.81 mL, 9.41 mmol) was dropwise added thereto at −20° C. and stirred overnight at −20° C. To the reaction mixture, saturated aqueous ammonium chloride solution was added dropwise at 0° C. to quench the reaction, and the mixture was extracted with ethyl acetate. The organic layer was washed with saturated brine and dried over anhydrous sodium sulfate. The residue obtained by removing the solvent was purified by column chromatography (silica gel, hexane/ethyl acetate) to afford the title compound (834 mg).

¹H-NMR (DMSO-d₆) δ (ppm) 6.94-6.74 (m, 1H), 5.61-5.36 (m, 1H), 3.97-3.78 (m, 1H), 3.12-2.92 (m, 2H), 1.82-1.78 (m, 3H), 1.77-1.66 (m, 2H), 1.39-1.33 (m, 9H), 1.12-1.07 (m, 9H).

### Third Step

### Production of tert-butyl (RS)-(3-aminohex-4-yn-1-yl)carbamate

A solution of tert-butyl ((RS)-3-(((RS)-tert-butylsulfinyl)amino)hex-4-yn-1-yl)carbamate (2.912 g, 9.2 mmol) in 4 M hydrogen chloride/1,4-dioxane (2.3 mL, 9.2 mmol) was stirred for one night at room temperature. Saturated sodium bicarbonate solution was added to the reaction mixture to quench the reaction, and the insoluble materials were filtered. The filtrate was extracted with ethyl acetate, and the organic layer was washed with saturated brine and then dried over anhydrous sodium sulfate. After the solvent was concentrated under reduced pressure, the residue was purified by column chromatography (silica gel, chloroform/methanol) to afford the title compound (1.363 g).

¹H-NMR (CDCl₃) δ (ppm) 5.08 (br, 1H), 3.64-3.54 (m, 1H), 3.42-3.28 (m, 1H), 3.28-3.15 (m, 1H), 1.81 (d, J=2.2 Hz, 3H), 1.77-1.70 (m, 2H), 1.44 (s, 9H).

### Reference Example 3

### Production of tert-butyl (RS)-(1-(1-aminobut-2-yn-1-yl)cyclopropyl)carbamate

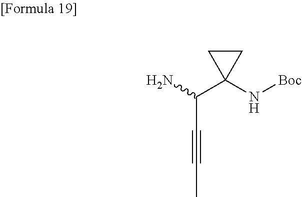 [SMILES: CC#CC(N)C1(NC(=O)OC(C)(C)C)CC1]

### First Step

### Production of tert-butyl (RS)-(1-(((tert-butylsulfinyl)imino)methyl)cyclopropyl)carbamate

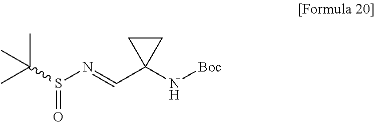 [SMILES: CC(C)(C)OC(=O)NC1(/C=N/S(=O)C(C)(C)C)CC1]

To a solution of tert-butyl (1-formylcyclopropyl)carbamate (1 g, 5.4 mmol) and 2-methyl-2-propanesulfinamide (1.309 g, 10.80 mmol) in THF (17.25 mL), tetraethoxytitanium (3.4 mL, 16.2 mmol) was added under a nitrogen flow and stirred overnight at room temperature. After saturated brine was added to the reaction mixture, the precipitated solid was collected by filtration, and the filtrate was extracted with ethyl acetate. The organic layer was washed with water and saturated brine and then dried over anhydrous sodium sulfate. The residue obtained by removing the solvent was purified by column chromatography (silica gel, hexane/ethyl acetate) to afford the title compound (1.308 g).

¹H-NMR (CDCl₃) δ (ppm) 7.47 (s, 1H), 5.29-4.89 (m, 1H), 1.52-1.07 (m, 22H); LC-MS (m/z): 289.4 [M+H]⁺.

### Second Step

### Production of tert-butyl (1-((RS)-(1-(((RS)-tert-butylsulfinyl)amino)but-2-yn-1-yl)cyclopropyl)carbamate

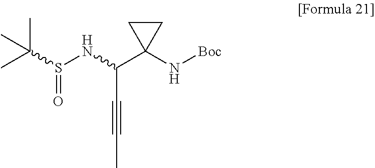 [SMILES: CC#CC(NS(=O)C(C)(C)C)C1(NC(=O)OC(C)(C)C)CC1]

To a solution of tert-butyl (1-(((tert-butylsulfinyl)imino)methyl)cyclopropyl)carbamate (1.3 g, 4.51 mmol) in THF (22.54 mL), 1-propynylmagnesium bromide in 0.5 M-THF solution (18.03 mL, 9.02 mmol) was added dropwise at −20° C. and stirred for 16 hours. Another 1-propynylmagnesium bromide in 0.5 M-THF solution (18.03 mL, 9.02 mmol) was dropwise added thereto at −20° C., stirred for 2.5 hours at the same temperature, and then stirred for 3 hours at room temperature. To the reaction mixture, saturated aqueous ammonium chloride solution was added dropwise at 0° C. to quench the reaction, and the mixture was further diluted with water and extracted with ethyl acetate. The organic layer was washed with water and saturated brine successively and dried over anhydrous sodium sulfate. Ethyl acetate and diisopropyl ether were added to the residue obtained by removing the solvent, and the precipitated solid was collected by filtration to afford the title compound (539 mg). The residue obtained by concentrating the filtrate was purified by column chromatography (silica gel, hexane/ethyl acetate) to afford the title compound (591 mg).

¹H-NMR (500 MHZ, DMSO-d₆) δ (ppm) 7.07 (s, 1H), 5.47-5.32 (m, 1H), 4.32-4.19 (m, 1H), 1.77 (d, J=2.3 Hz, 3H), 1.36 (s, 9H), 1.12 (s, 9H), 1.01-0.80 (m, 2H), 0.72-0.50 (m, 2H); LC-MS (m/z): 329.3 [M+H]⁺.

### Third Step

### Production of tert-butyl (RS)-(1-(1-aminobut-2-yn-1-yl)cyclopropyl)carbamate

To a solution of tert-butyl (1-(1-((tert-butylsulfinyl)amino)but-2-yn-1-yl)cyclopropyl)carbamate (530 mg, 1.614 mmol) in 1,4-dioxane (16.135 mL), 4 M hydrogen chloride/1,4-dioxane solution (0.323 mL, 1.291 mmol) was added after the 4 M hydrogen chloride/1,4-dioxane solution (0.323 mL, 1.291 mmol) was stirred for 2.5 hours at room temperature, and stirred for 2 days at room temperature. To complete the reaction, another 4 M hydrogen chloride/1,4-dioxane solution (0.323 mL, 1.291 mmol) was added thereto and stirred for 3 hours at room temperature. Saturated sodium bicarbonate solution was added to the reaction mixture to quench the reaction, and the insoluble materials were filtered. The filtrate was extracted with ethyl acetate, and the organic layer was washed with water and saturated brine successively and then dried over anhydrous sodium sulfate. The solvent was concentrated under reduced pressure to afford a reaction mixture. The reaction mixture was combined with a reaction mixture obtained by repeating this procedure, and the combined mixture was purified by column chromatography (silica gel, chloroform/methanol) to afford the title compound (686 mg).

¹H-NMR (CDCl₃) δ (ppm) 5.10 (br, 1H), 3.65 (d, J=2.2 Hz, 1H), 1.79 (d, J=2.3 Hz, 3H), 1.44 (s, 9H), 1.08-0.64 (m, 4H); LC-MS (m/z): 225.1 [M+H]⁺.

### Reference Example 4

### Production of 5-isothiocyanato-2,3-dihydrobenzo[b][1,4]dioxin

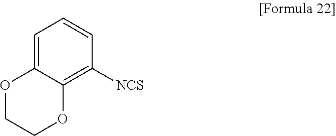

To 2,3-dihydrobenzo[b][1,4]dioxin-5-amine (41 g, 271.52 mmol) in aqueous solution (700 mL), thiophosgene (50.6 mL, 678.8 mmol) was added at 0° C., and the mixture was stirred for 1 hour at room temperature. After completion of the reaction, the reaction mixture was extracted with ethyl acetate. The organic layer was washed with saturated brine and then dried over anhydrous sodium sulfate. After the solvent was concentrated under reduced pressure, the residue was purified by column chromatography (silica gel, petroleum ether) to afford the title compound (yield: 40 g).

¹H-NMR (CDCl₃) δ (ppm) 6.86-6.64 (m, 3H), 4.40-4.34 (m, 2H), 4.30-4.25 (m, 2H); LC-MS (m/z): 194.1 [M+H]⁺.

### Example 1

### Production of (RS)-1-(7,8-dihydro-[1,4]dioxino[2′,3′:5,6]benzo[1,2-d]thiazol-2-yl)-6-(prop-1-yn-1-yl)tetrahydropyrimidin-2(1H)-one

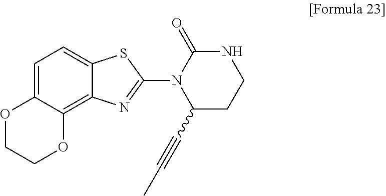 [SMILES: CC#CC1CCNC(=O)N1c1nc2c3c(ccc2s1)OCCO3]

### First Step

### Production of tert-butyl (RS)-(3-(3-(2,3-dihydrobenzo[b][1,4]dioxin-5-yl)thioureido)hex-4-yn-1-yl)carbamate

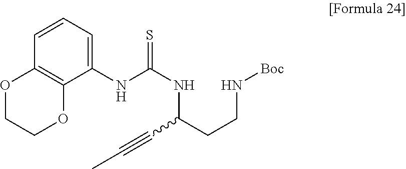 [SMILES: CC#CC(CCNC(=O)OC(C)(C)C)NC(=S)Nc1cccc2c1OCCO2]

To a solution of tert-butyl (RS)-(3-aminohex-4-yn-1-yl)carbamate (Reference Example 2) (0.5 g, 2.355 mmol) in THF (21.41 mL), 5-isothiocyanato-2,3-dihydrobenzo[b][1,4]dioxin (Reference Example 4) (0.414 g, 2.141 mmol) and TEA (0.328 mL, 2.355 mmol) were added and stirred overnight at room temperature. The reaction mixture was diluted by adding water thereto and extracted with ethyl acetate. The organic layer was washed with water and saturated brine successively and dried over anhydrous sodium sulfate, then the residue obtained by removing the solvent was purified by column chromatography (silica gel, hexane/ethyl acetate) to afford the title compound (0.79 g).

¹H-NMR (DMSO-d₆) δ (ppm) 8.97 (s, 1H), 8.15 (d, J=8.5 Hz, 1H), 7.34 (d, J=8.0 Hz, 1H), 6.82-6.78 (m, 1H), 6.77-6.72 (m, 1H), 6.70-6.65 (m, 1H), 5.25 (br, 1H), 4.36-4.17 (m, 4H), 3.09-2.94 (m, 2H), 1.84-1.66 (m, 5H), 1.37 (s, 9H); LC-MS (m/z): 406.2 [M+H]⁺.

### Second Step

### Production of tert-butyl (RS)-(3-((7,8-dihydro-[1,4]dioxino[2′,3′:5,6]benzo[1,2-d]thiazol-2-yl)amino)hex-4-yn-1-yl)carbamate

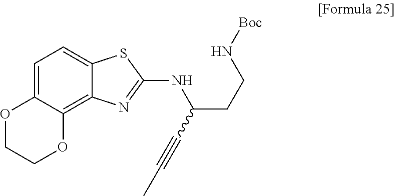 [SMILES: CC#CC(CCNC(=O)OC(C)(C)C)Nc1nc2c3c(ccc2s1)OCCO3]

To a solution of tert-butyl (RS)-(3-(3-(2,3-dihydrobenzo[b][1,4]dioxin-5-yl)thioureido)hex-4-yn-1-yl)carbamate (0.785 g, 1.936 mmol) and sodium hydrogencarbonate (3.25 g, 38.7 mmol) in chloroform (64.5 mL), benzyltrimethylammonium tribromide (0.679 g, 1.742 mmol) was added slowly at 0° C. and stirred for 5 minutes. Saturated brine was added to the reaction mixture at 0° C. to quench the reaction, and the mixture was extracted with chloroform. After the organic layer was washed with saturated brine and then dried over anhydrous sodium sulfate, the residue obtained by removing the solvent was purified by column chromatography (silica gel, chloroform) to afford the title compound (173 mg).

¹H-NMR (CDCl₃) δ (ppm) 7.02 (d, J=8.6 Hz, 1H), 6.70 (d, J=8.6 Hz, 1H), 5.44 (br, 1H), 5.27 (br, 1H), 4.73 (br, 1H), 4.44-4.36 (m, 2H), 4.33-4.28 (m, 2H), 3.48-3.26 (m, 1H), 3.25-3.11 (m, 1H), 2.08-1.84 (m, 2H), 1.81 (d, J=2.3 Hz, 3H), 1.46 (s, 9H); LC-MS (m/z): 404.2 [M+H]⁺.

### Third Step

### Production of (RS)-1-(7,8-dihydro-[1,4]dioxino[2′,3′:5,6]benzo[1,2-d]thiazol-2-yl)-6-(prop-1-yn-1-yl)tetrahydropyrimidin-2(1H)-one

A solution of tert-butyl (RS)-(3-((7,8-dihydro-[1,4]dioxino[2′,3′:5,6]benzo[1,2-d]thiazol-2-yl)amino)hex-4-yn-1-yl)carbamate (0.167 g, 0.414 mmol) and potassium carbonate (0.572 g, 4.14 mmol) in DMF (7 mL) was stirred for 6 days at 110° C. After cooling to room temperature, the reaction mixture was diluted with water and extracted with ethyl acetate. The organic layer was washed with water and saturated brine successively and dried over anhydrous sodium sulfate, then the residue obtained by removing the solvent was purified by column chromatography (silica gel, hexane/ethyl acetate) to afford a crude product. Chloroform and diisopropyl ether were added to the resulting crude product, and the precipitated solid was collected by filtration to afford the title compound (73 mg).

¹H-NMR (DMSO-d₆) δ (ppm) 7.73-7.65 (m, 1H), 7.26 (d, J=8.5 Hz, 1H), 6.81 (d, J=8.5 Hz, 1H), 5.88-5.78 (m, 1H), 4.44-4.24 (m, 4H), 3.60-3.48 (m, 1H), 3.32-3.24 (m, 1H), 2.16-2.04 (m, 2H), 1.80 (d, J=2.2 Hz, 3H); LC-MS (m/z): 329.9 [M+H]⁺.

### Example 2

### Production of (RS)-6-(7,8-dihydrobenzofuro[4,5-d]thiazol-2-yl)-7-(prop-1-yn-1-yl)-4,6-diazaspiro[2.4]heptan-5-one

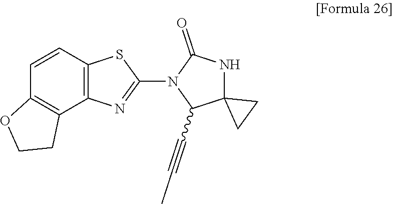 [SMILES: CC#CC1N(c2nc3c4c(ccc3s2)OCC4)C(=O)NC12CC2]

### First Step

### Production of tert-butyl (RS)-(1-(1-(3-(2,3-dihydrobenzofuran-4-yl)thioureido)but-2-yn-1-yl)cyclopropyl)carbamate

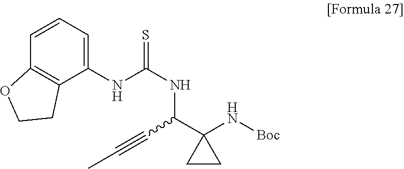 [SMILES: CC#CC(NC(=S)Nc1cccc2c1CCO2)C1(NC(=O)OC(C)(C)C)CC1]

To a solution of 4-isothiocyanato-2,3-dihydrobenzofuran (Reference Example 1) (0.158 g, 0.892 mmol) in THF (4.46 mL), a solution of tert-butyl (RS)-1-(1-aminobut-2-yn-1-yl)cyclopropyl)carbamate (Reference Example 3) (0.2 g, 0.892 mmol) in THE (2 mL) was added and stirred for 2 hours at room temperature. The residue obtained by removing the solvent was purified by column chromatography (silica gel, hexane/ethyl acetate) to afford the title compound (306 mg).

¹H-NMR (CDCl₃) δ (ppm) 8.05 (br, 1H), 7.43 (br, 1H), 7.18-7.10 (m, 1H), 6.78 (d, J=7.9 Hz, 1H), 6.73 (d, J=8.0 Hz, 1H), 5.11 (br, 1H), 4.85 (s, 1H), 4.68-4.53 (m, 2H), 3.32-3.14 (m, 2H), 1.85 (d, J=2.2 Hz, 3H), 1.29 (s, 9H), 1.06-0.74 (m, 4H); LC-MS (m/z): 402.2 [M+H]⁺.

### Second Step

### Production of tert-butyl (RS)-(1-(1-((7,8-dihydrobenzofuro[4,5-d]thiazol-2-yl)amino)but-2-yn-1-yl)cyclopropyl)carbamate

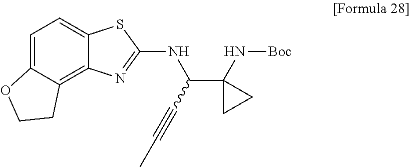 [SMILES: CC#CC(Nc1nc2c3c(ccc2s1)OCC3)C1(NC(=O)OC(C)(C)C)CC1]

To a solution of tert-butyl (RS)-(1-(1-(3-(2,3-dihydrobenzofuran-4-yl)thioureido)but-2-yn-1-yl)cyclopropyl)carbamate (0.3 g, 0.747 mmol) and sodium hydrogencarbonate (0.628 g, 7.47 mmol) in chloroform (25 mL), benzyltrimethylammonium tribromide (0.262 g, 0.672 mmol) was added slowly at 0° C. and stirred for 6 minutes. Water was added to the reaction mixture at 0° C. to quench the reaction, and the mixture was extracted with chloroform. After the organic layer was washed with saturated brine and then dried over anhydrous sodium sulfate, the residue obtained by removing the solvent was purified by column chromatography (silica gel, hexane/ethyl acetate) to afford the title compound (259 mg).

¹H-NMR (CDCl₃) δ (ppm) 7.30-7.27 (m, 1H), 6.67-6.52 (m, 2H), 5.16 (s, 1H), 4.63 (t, J=8.7 Hz, 2H), 4.61-4.56 (m, 1H), 3.46-3.36 (m, 2H), 1.81 (d, J=2.2 Hz, 3H), 1.43 (s, 9H), 1.23-1.15 (m, 1H), 1.09-1.01 (m, 1H), 0.99-0.92 (m, 1H), 0.87-0.79 (m, 1H); LC-MS (m/z): 400.2 [M+H]⁺.

### Third Step

### Production of (RS)-6-(7,8-dihydrobenzofuro[4,5-d]thiazol-2-yl)-7-(prop-1-yn-1-yl)-4,6-diazaspiro[2.4]heptan-5-one

A solution of tert-butyl (RS)-(1-(1-((7,8-dihydrobenzofuro[4,5-d]thiazol-2-yl)amino)but-2-yn-1-yl)cyclopropyl)carbamate (0.1 g, 0.25 mmol) and potassium carbonate (0.173 g, 1.252 mmol) in DMF (2.5 mL) was stirred overnight at 110° C. After cooling to room temperature, the reaction mixture was diluted with water, and the precipitated solid was collected by filtration to afford the title compound (44 mg).

¹H-NMR (DMSO-d₆) δ (ppm) 8.18 (s, 1H), 7.67-7.58 (m, 1H), 6.77 (d, J=8.4 Hz, 1H), 5.22-5.15 (m, 1H), 4.67-4.57 (m, 2H), 3.36 (t, J=8.5 Hz, 2H), 1.81 (d, J=2.1 Hz, 3H), 1.16-1.04 (m, 1H), 1.02-0.89 (m, 2H), 0.88-0.79 (m, 1H); LC-MS (m/z): 325.9 [M+H]⁺.

Each of the compounds of Examples 3 to 9, and 16 to 23 in the following [Table 1] and [Table 3] were produced with corresponding raw materials (commercially available products or compounds derivatized from commercially available compounds by a known method or an equivalent method thereof) according to the method described in the above Examples, if necessary, by using a method which is usually used in organic synthetic chemistry in combination. The physicochemical data of each compound is shown in [Table 2] and [Table 4]. In the tables, when the bond of the substituent at an optically active center of a compound is indicated by a wavy line, it indicates that the compound is a mixture of R and S bodies with respect to the stereochemistry at a substitution position thereof; and when the bond of the substituent at an optically active center is indicated by a solid line, it indicates that the compound is an R or S form for a substitution position thereof. Each enantiomer was obtained appropriately as a single compound by optical resolution by supercritical fluid chromatography using a chiral column.

TABLE 1

| Example | | |
|---|---|---|
| Number | Structure | Compound Name |
| 3 | 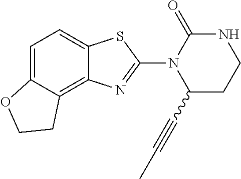 [SMILES: CC#CC1CCNC(=O)N1c1nc2c3c(ccc2s1)OCC3] | (RS)-3-(7,8-dihydrobenzofuro[4,5- d]thiazol-2-yl)-4-(prop-1-yn-1- yl)-tetrahydropyrimidin-2(1H)-one |
| 4 | 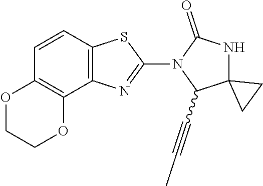 [SMILES: CC#CC1N(c2nc3c4c(ccc3s2)OCCO4)C(=O)NC12CC2] | (RS)-6-(7,8-dihydro- [1,4]dioxyno[2′,3′:5,6]benzo[1,2- d]thiazol-2-yl)-7-(prop-1-yn-1- yl)-4,6-diazaspiro[2.4]heptan-5- one |
| 5 (Enantiomer A of Ex. 1) | 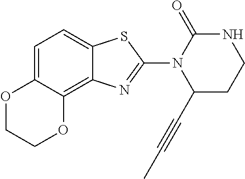 [SMILES: CC#CC1CCNC(=O)N1c1nc2c3c(ccc2s1)OCCO3] | 1-(7,8-dihydro- [1,4]dioxyno[2′,3′:5,6]benzo[1,2- d]thiazol-2-yl)-6-(prop-1-yn-1- yl)tetrahydropyrimidin-2(1H)-one |
| 6 (Enantiomer B of Ex. 1) | 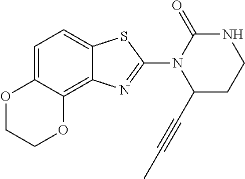 [SMILES: CC#CC1CCNC(=O)N1c1nc2c3c(ccc2s1)OCCO3] | 1-(7,8-dihydro- [1,4]dioxyno[2′,3′:5,6]benzo[1,2- d]thiazol-2-yl)-6-(prop-1-yn-1- yl)tetrahydropyrimidin-2(1H)-one |
| 7 (Enantiomer A of Ex. 4) | 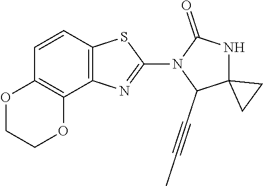 [SMILES: CC#CC1N(c2nc3c4c(ccc3s2)OCCO4)C(=O)NC12CC2] | 6-(7,8-dihydro- [1,4]dioxyno[2′,3′:5,6]benzo[1,2- d]thiazol-2-yl)-7-(prop-1-yn-1- yl)-4,6-diazaspiro[2.4]heptan-5- one |
| 8 (Enantiomer B of Ex. 4) | 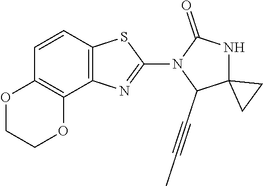 [SMILES: CC#CC1N(c2nc3c4c(ccc3s2)OCCO4)C(=O)NC12CC2] | 6-(7,8-dihydro- [1,4]dioxyno[2′,3′:5,6]benzo[1,2- d]thiazol-2-yl)-7-(prop-1-yn-1- yl)-4,6-diazaspiro[2.4]heptan-5- one |
| 9 | 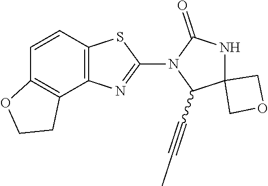 [SMILES: CC#CC1N(c2nc3c4c(ccc3s2)OCC4)C(=O)NC12COC2] | (RS)-7-(7,8-dihydro- benzofuro[4,5-d]thiazol-2-yl)-8- (prop-1-yn-1-yl)-2-oxa-5,7- diazaspiro[3.4]octan-6-one |

TABLE 2

| | | LCMS m/z |
|---|---|---|
| Ex. No. | ¹H NMR δ (ppm) | [M + H] [symbol] |
| 3 | (DMSO-d6) δ 7.75-7.69 (m, 1H), 7.61-7.52 (m, | 313.9 |
| | 1H), 6.75 (d, J = 8.4 Hz, 1H), 5.89-5.82 (m, 1H), | |
| | 4.62 ( [symbol] J = 8.7 Hz, 2H), 3.60-3.49 (m, 1H), 3.44- | |
| | 3.38 (m, 3H) [symbol] 2.15-2.06 (m, 2H), 1.79 (d, J = 2.2 | |
| | Hz, [symbol] H). | |
| 4 | (DMSO-d6) δ 8.15 (s, 1H), 7.31 (d, J = 8.6 Hz, 1H), | 341.8 |
| | 6.83 (d, J = 8.6 Hz, 1H), 5.17 (d, J = 2.2 Hz, 1H), | |
| | 4.43-4.26 (m, 4H), 1.81 (d, J = 2.1 Hz, 3H), 1.14- | |
| | 1.04 (m, 1H), 1.00-0.91 (m, 2H), 0.89-0.79 (m, 1H) | |
| 5 | (DMSO-d6) δ 7.75-7.60 (m, 1H), 7.26 (d, J = 8.5 | 330.1 |
| | Hz, 1H), 6.81 (d, J [symbol] 8.5 Hz, 1H), 5.89-5.75 (m, | |
| | 1H), 4.48-4.22 (m, 4H), 3.64-3.46 (m, 1H), 3.35- | |
| | 3.22 (m, 1H), 2.21-2.00 (m, 2H), 1.80 (d, J = 2.2 | |
| | Hz, 3H). | |
| 6 | (DMSO-d6) δ 7.70-7.64 (m, 1H), 7.24 (d, J = 8.5 | 330.2 |
| | Hz, 1H), 6.79 (d, J = 8.5 Hz, 1H), 5.84-5.78 (m, 1H), | |
| | 4.42-4.22 (m, 4H), 3.59-3.47 (m, 1H), 3.40- | |
| | 3.18 (m, 1H), 2.14-2.03 (m, 2H), 1.78 (d, J = 2.2 | |
| | Hz, 3H). | |
| 7 | (DMSO-d6) δ = 8.17 (br s, 1H), 7.31 (d, J = 8.8 Hz, | 342.2 |
| | 1H), 6.82 (d, J = 8.4 Hz, 1H), 5.15-5.12 (m, 1H), | |
| | 4.40-4.30 (m, 4H), 1.81 (d, J = 2.0 Hz, 3H), 1.11-1.05 | |
| | (m, 1H), 0.95 (d, J = 8.6 Hz [symbol] 2H), 0.86-0.81 (m, 1H). | |
| 8 | (DMSO-d6) δ = 8.14 (br s, 1H), 7.30 (d, J = 8.4 Hz, | 342.2 |
| | 1H), 6.82 (d, J = 8.4 Hz, 1H), 5.15-5.12 (m, 1H), | |
| | 4.40-4.30 (m, 4H), 1.81 (d, J = 2.0 Hz, 3H), 1.08-1.05 | |
| | (m, 1H), 0.94 (d, J = 8.6 Hz, 2H), 0.86-0.81 (m, 1H). | |
| 9 | (DMSO-d6) δ 9.09 (s, 1H), 7.68-7.56 (m, 1H), 6.85-6.72 | 342.1 |
| | (m, 1H), 5.70-5.60 (m, 1H), 4.98 (d, J = 7.5 Hz, 1H), | |
| | 4.81 (d, J = 7.1 Hz, 1H), 4.72-4.59 (m, 3H), 4.56 (d, J = | |
| | 7.0 Hz, 1H, 3.44-3.36 (m, 2H), 1.84 (d, J = 2.2 Hz, 3H). | |

[symbol]

indicates data missing or illegible when filed

### Example 10

### Production of (5R*,6S*)-1-(7,8-dihydrobenzofuro[4,5-d]thiazol-2-yl)-5-methyl-6-(prop-1-yn-1-yl)tetrahydropyrimidin-2(1H)-one (Relative Configuration)

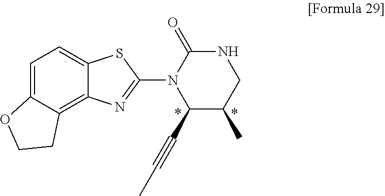 [SMILES: CC#C[C@@H]1[C@H](C)CNC(=O)N1c1nc2c3c(ccc2s1)OCC3]

### First Step

### Production of tert-butyl (RS)-{3-[methoxy(methyl)amino]-2-methyl-3-oxopropyl}carbamate

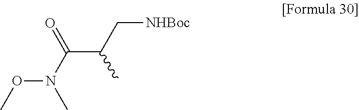 [SMILES: CON(C)C(=O)C(C)CNC(=O)OC(C)(C)C]

To a solution of (RS)-3-[(tert-butoxycarbonyl)amino]-2-methylpropanoic acid (33 g, 162.56 mmol) in DCM (500 mL), N, O-dimethylhydroxylamine hydrochloride (17.34 g, 178.82 mmol), N-methylmorpholine (20 mL, 178.82 mmol), and 1-(3-dimethylaminopropyl)-3-ethylcarbodiimide hydrochloride (34.15 g, 178.82 mmol) were added at 0° C. and stirred for 3 hours at room temperature. The reaction mixture was diluted with DCM, washed with water and saturated brine successively, and then dried over anhydrous sodium sulfate. The solvent was removed to afford the title compound (43 g) as a crude product.

¹H NMR (DMSO-d₆) δ=3.65 (s, 3H), 3.55 (t, J=4.2 Hz, 1H), 3.09-3.01 (m, 5H), 2.95-2.90 (m, 1H), 1.36 (s, 9H), 0.95 (d, J=6.4 Hz, 3H). LC-MS (m/z): 247.23 [M+H]⁺.

### Second Step

### Production of tert-butyl (RS)-(2-methyl-3-oxohex-4-yn-1-yl)carbamate

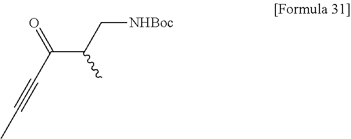 [SMILES: CC#CC(=O)C(C)CNC(=O)OC(C)(C)C]

To a solution of tert-butyl (RS)-{3-[methoxy(methyl)amino]-2-methyl-3-oxopropyl}carbamate (25 g, 101.63 mmol) in THE (200 mL), 1-propynylmagnesium bromide in 0.5 M-THF solution (610 mL, 304.87 mmol) was added dropwise at −60° C. and then stirred for 16 hours at room temperature. Water was added to the reaction mixture and extracted with ethyl acetate. The organic layer was washed with saturated brine and dried over anhydrous sodium sulfate. The residue obtained by removing the solvent was purified by column chromatography (silica gel, petroleum ether/ethyl acetate) to afford the title compound (7 g).

¹H NMR (500 MHZ, CDCl₃) δ=4.85 (br. s, 1H), 3.39-3.25 (m, 2H), 2.83-2.74 (m, 1H), 2.07 (s, 3H), 1.43 (s, 9H), 1.21 (d, J=7.3 Hz, 3H). LC-MS (m/z): 226.13 [M+H]⁺.

### Third Step

### Production of tert-butyl {(2RS)-3-[((R)-tert-butylsulfinyl)amino]-2-methylhex-4-yn-1-yl}carbamate

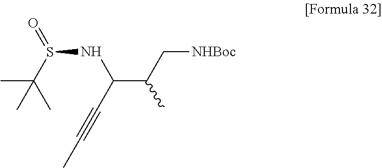 [SMILES: CC#CC(N[S@](=O)C(C)(C)C)C(C)CNC(=O)OC(C)(C)C]

To a solution of tert-butyl (RS)-(2-methyl-3-oxohex-4-yn-1-yl)carbamate (1.5 g, 6.66 mmol) in toluene (20 mL), (R)-(+)-tert-butylsulfinamide (0.967 g, 7.99 mmol) and tetraethoxytitanium (16.41 mL, 77.78 mmol) were added at 0° C. and stirred for 8 hours at 80° C. Sodium borohydride (2.24 g, 62.22 mmol) was added to the reaction solution at −30° C. and stirred for another 10 hours at room temperature. Aqueous methanol solution was added dropwise to the reaction solution at 0° C. to quench the reaction, and the mixture was filtered by celite. The filtrate was diluted with DCM, washed with water and saturated brine successively, and then dried over anhydrous sodium sulfate. After the solvent was removed, the residue was purified by column chromatography (silica gel, petroleum ether/ethyl acetate) to afford the title compound (0.92 g).

1H NMR (DMSO-d₆) δ=6.98-6.69 (m, 1H), 5.25 (dd, J=5.6, 17.1 Hz, 1H), 4.02-3.83 (m, 1H), 3.22-2.64 (m, 2H), 1.95-1.87 (m, 1H), 1.82 (s, 3H), 1.38 (s, 9H), 1.20 (s, 9H), 0.92-0.81 (m, 3H). LC-MS (m/z): 331.39 [M+H]⁺.

### Fourth Step

### Production of tert-butyl ((2RS)-3-amino-2-methylhex-4-yn-1-yl)carbamate

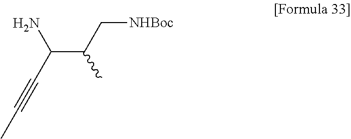 [SMILES: CC#CC(N)C(C)CNC(=O)OC(C)(C)C]

To tert-butyl {(2RS)-3-[((R)-tert-butylsulfinyl)amino]-2-methylhex-4-yn-1-yl}carbamate (0.92 g, 2.78 mmol) in 1,4-dioxane (10 mL), 4 M hydrogen chloride/1,4-dioxane solution (0.69 mL, 2.78 mmol) was added at 0° C. and stirred for 16 hours at room temperature. Saturated sodium bicarbonate solution were added to the reaction mixture, the mixture was extracted with ethyl acetate, and the organic layer was washed with water and saturated brine successively and then dried over anhydrous sodium sulfate. The solvent was concentrated under reduced pressure to afford the title compound (0.57 g).

1H NMR (DMSO-d₆) δ=6.82 (s, 1H), 3.44-3.35 (m, 2H), 3.08-2.89 (m, 1H), 2.87-2.75 (m, 1H), 1.91-1.82 (m, 1H), 1.77 (s, 3H), 1.65-1.51 (m, 1H), 1.37 (s, 9H), 0.84 (dd, J=3.9, 6.6 Hz, 3H).

### Fifth Step

### Production of tert-butyl {(2RS)-3-[3-(2,3-dihydrobenzofuran-4-yl)thioureido]-2-methylhex-4-yn-1-yl}carbamate

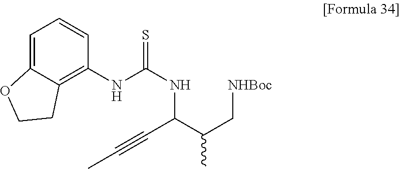 [SMILES: CC#CC(NC(=S)Nc1cccc2c1CCO2)C(C)CNC(=O)OC(C)(C)C]

To a solution of tert-butyl ((2RS)-3-amino-2-methylhex-4-yn-1-yl)carbamate (0.57 g, 2.52 mmol) in THE (10 mL), TEA (1.06 mL, 7.57 mmol) and 4-isothiocyanato-2,3-dihydrobenzofuran (Reference Example 1) (0.446 g, 2.52 mmol) were added and stirred for 16 hours at room temperature. After completion of the reaction, the reaction mixture was diluted with water and extracted with ethyl acetate. The organic layer was washed with saturated brine and dried over anhydrous sodium sulfate. After the solvent was removed, the resulting residue was purified by column chromatography (silica gel, petroleum ether/ethyl acetate) to afford the title compound (0.7 g).

¹H NMR (500 MHZ, DMSO-d₆) δ=9.12 (d, J=16.5 Hz, 1H), 8.11-8.05 (m, 1H), 7.17-7.09 (m, 1H), 7.04 (t, J=8.1 Hz, 1H), 6.84-6.74 (m, 1H), 6.58 (dd, J=3.8, 7.8 Hz, 1H), 5.29-5.18 (m, 1H), 4.56-4.45 (m, 2H), 3.18-2.93 (m, 3H), 2.89-2.75 (m, 1H), 1.98-1.93 (m, 1H), 1.84-1.80 (m, 3H), 1.37 (s, 9H), 0.90 (d, J=6.7 Hz, 3H). LC-MS (m/z): 404.25 [M+H]⁺.

### Sixth Step

### Production of tert-butyl {(2RS)-3-(7,8-dihydrobenzofuro[4,5-d]thiazol-2-yl)amino]-2-methylhex-4-yn-1-yl}carbamate

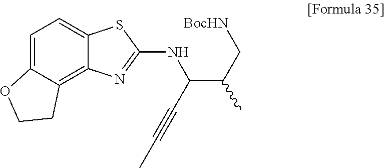 [SMILES: CC#CC(Nc1nc2c3c(ccc2s1)OCC3)C(C)CNC(=O)OC(C)(C)C]

To a solution of tert-butyl {(2RS)-3-[3-(2,3-dihydrobenzofuran-4-yl)thioureido]-2-methylhex-4-yn-1-yl}carbamate (0.7 g, 1.74 mmol) in chloroform (10 mL), sodium hydrogencarbonate (1.45 g, 17.37 mmol) and benzyltrimethylammonium tribromide (0.609 g, 1.56 mmol) were added at 0° C. and stirred for 2 hours. Water was added to the reaction mixture to quench the reaction, and the mixture was extracted with DCM. After the organic layer was washed with saturated brine and then dried over anhydrous sodium sulfate, the solvent was removed to afford the title compound (0.7 g) as a crude product. LC-MS (m/z): 402.22 [M+H]⁺.

### Seventh Step

### Production of (2RS)—N3-(7,8-dihydrobenzofuro[4,5-d]thiazol-2-yl)-2-methylhex-4-yn-1,3-diamine

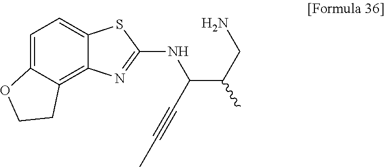 [SMILES: CC#CC(Nc1nc2c3c(ccc2s1)OCC3)C(C)CN]

To tert-butyl {(2RS)-3-[(7,8-dihydrobenzofuro[4,5-d]thiazol-2-yl)amino]-2-methylhex-4-yn-1-yl}carbamate (0.7 g, 1.74 mmol) in 1,4-dioxane (10 mL), 4 M hydrogen chloride/1,4-dioxane solution (5 mL) was added at 0° C. and stirred for 2 hours at room temperature. Toluene was added to the residue obtained by removing the solvent, and the remaining water was removed by toluene azeotrope to afford the title compound (0.7 g) as a crude product. LC-MS (m/z): 302.38 [M+H]⁺.

### Eighth Step

### Production of (5R*,6S*)-1-(7,8-dihydrobenzofuro[4,5-d]thiazol-2-yl)-5-methyl-6-(prop-1-yn-1-yl)tetrahydropyrimidin-2(1H)-one (Relative Configuration)

To a solution of (2RS)—N3-(7,8-dihydrobenzofuro[4,5-d]thiazol-2-yl)-2-methylhex-4-yn-1,3-diamine (0.7 g, 2.32 mmol) in DMF (10 mL), TEA (0.97 mL, 6.98 mmol) and N,N′-disuccinimidyl carbonate (0.595 g, 2.32 mmol) were added at 0° C. and stirred for 16 hours at room temperature. After water was added to the reaction mixture, the precipitated solid was collected by filtration to afford a crude product of the diastereomeric mixture of cis-1-(7,8-dihydrobenzofuro[4,5-d]thiazol-2-yl)-5-methyl-6-(prop-1-yn-1-yl)tetrahydropyrimidin-2(1H)-one and trans-1-(7,8-dihydrobenzofuro[4,5-d]thiazol-2-yl)-5-methyl-6-(prop-1-yn-1-yl)tetrahydropyrimidin-2(1H)-one. The resulting diastereomeric mixture was purified by HPLC preparative chromatography to afford a cis-form of the titled compound (90 mg) as a fraction eluted later (retention time: 1.93 minutes).

¹H NMR (DMSO-d₆) δ=7.70 (d, J=3.7 Hz, 1H), 7.57 (d, J=8.6 Hz, 1H), 6.75 (d, J=8.3 Hz, 1H), 5.66 (br. s, 1H), 4.62 (t, J=8.8 Hz, 2H), 3.43-3.36 (m, 2H), 3.21-3.10 (m, 2H), 2.37-2.28 (m, 1H), 1.81 (d, J=2.0 Hz, 3H), 1.11 (d, J=6.6 Hz, 3H). LC-MS (m/z): 328.17 [M+H]⁺.

### Example 11

### Production of (5S*, 6S*)-1-(7,8-dihydrobenzofuro[4,5-d]thiazol-2-yl)-5-methyl-6-(prop-1-yn-1-yl)tetrahydropyrimidin-2(1H)-one (Relative Configuration)

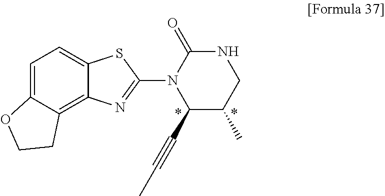 [SMILES: CC#C[C@@H]1[C@@H](C)CNC(=O)N1c1nc2c3c(ccc2s1)OCC3]

The diastereomeric mixture obtained in the eighth step of Example 10 was purified by HPLC preparative chromatography to afford a trans-form of the titled compound (70 mg) as a fraction eluted first (retention time: 1.90 minutes).

¹H NMR (DMSO-d₆) δ=7.64 (d, J=4.2 Hz, 1H), 7.57 (d, J=8.3 Hz, 1H), 6.76 (d, J=8.3 Hz, 1H), 5.57-5.52 (m, 1H), 4.62 (t, J=8.8 Hz, 2H), 3.71 (dd, J=4.4, 12.0 Hz, 1H), 3.39 (t, J=8.7 Hz, 2H), 3.04-2.97 (m, 1H), 2.40-2.33 (m, 1H), 1.80 (d, J=2.2 Hz, 3H), 1.03 (d, J=6.8 Hz, 3H). LC-MS (m/z): 328.20 [M+H]⁺.

### Example 12

### Production of (5S*,6R*)-1-(7,8-dihydrobenzofuro[4,5-d]thiazol-2-yl)-5-methyl-6-(prop-1-yn-1-yl)tetrahydropyrimidin-2(1H)-one (Relative Configuration)

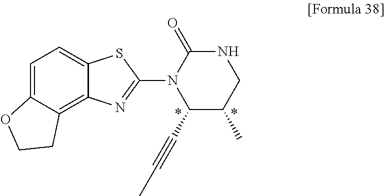 [SMILES: CC#C[C@H]1[C@@H](C)CNC(=O)N1c1nc2c3c(ccc2s1)OCC3]

A diastereomer mixture of cis-1-(7,8-dihydrobenzofuro[4,5-d]thiazol-2-yl)-5-methyl-6-(prop-1-yn-1-yl)tetrahydropyrimidin-2(1H)-one and trans-1-(7,8-dihydrobenzofuro[4,5-d]thiazol-2-yl)-5-methyl-6-(prop-1-yn-1-yl)tetrahydropyrimidin-2(1H)-one was obtained by the synthesis in the same manner as the compound of Example 10 by using(S)-(−)-tert-butylsulfinamide instead of (R)-(+)-tert-butylsulfinamide used in the third step of Example 10. The resulting diastereomeric mixture was purified by HPLC preparative chromatography to afford a cis-form of the titled compound (500 mg) as a fraction eluted later (retention time: 1.94 minutes).

¹H NMR (DMSO-d₆) δ=7.69 (d, J=3.7 Hz, 1H), 7.57 (d, J=8.6 Hz, 1H), 6.75 (d, J=8.6 Hz, 1H), 5.66 (br. s, 1H), 4.62 (t, J=8.8 Hz, 2H), 3.39 (dt, J=1.8, 8.7 Hz, 2H), 3.20-3.11 (m, 2H), 2.36-2.28 (m, 1H), 1.82 (d, J=2.2 Hz, 3H), 1.11 (d, J=6.6 Hz, 3H). LC-MS (m/z): 328.28 [M+H]⁺.

### Example 13

### Production of (5R*,6R*)-1-(7,8-dihydrobenzofuro[4,5-d]thiazol-2-yl)-5-methyl-6-(prop-1-yn-1-yl)tetrahydropyrimidin-2(1H)-one (Relative Configuration)

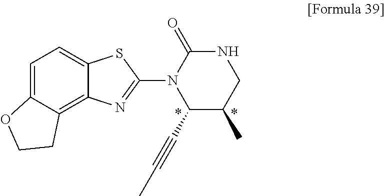 [SMILES: CC#C[C@H]1[C@H](C)CNC(=O)N1c1nc2c3c(ccc2s1)OCC3]

The diastereomeric mixture obtained in Example 12 was purified by HPLC preparative chromatography to afford a trans-form of the titled compound (330 mg) as a fraction eluted first (retention time: 1.91 minutes).

¹H NMR (DMSO-d₆) δ=7.64 (d, J=3.9 Hz, 1H), 7.57 (d, J=8.3 Hz, 1H), 6.76 (d, J=8.6 Hz, 1H), 5.58-5.53 (m, 1H), 4.62 (t, J=8.8 Hz, 2H), 3.71 (dd, J=4.2, 12.0 Hz, 1H), 3.39 (t, J=8.7 Hz, 2H), 3.03-2.96 (m, 1H), 2.39-2.33 (m, 1H), 1.80 (d, J=2.2 Hz, 3H), 1.03 (d, J=7.1 Hz, 3H). LC-MS (m/z): 328.28 [M+H]⁺.

### Example 14

### Production of (4R,6R)-1-(7,8-dihydrobenzofuro[4,5-d]thiazol-2-yl)-4-methyl-6-(prop-1-yn-1-yl)tetrahydropyrimidin-2(1H)-one

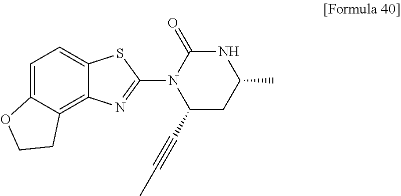 [SMILES: CC#C[C@H]1C[C@@H](C)NC(=O)N1c1nc2c3c(ccc2s1)OCC3]

### First Step

### Production of tert-butyl {(2R,E)-4-[(tert-butylsulfinyl)imino]butan-2-yl}carbamate

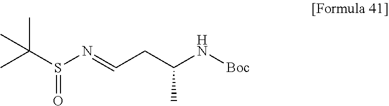 [SMILES: C[C@H](C/C=N/S(=O)C(C)(C)C)NC(=O)OC(C)(C)C]

To a solution of tert-butyl (R)-(4-oxobutan-2-yl)carbamate (9 g, 48.13 mmol) in THE (90 mL), tert-butylsulfinamide (8.73 g, 72.19 mmol) and tetraethoxytitanium (20.32 mL, 96.25 mmol) were added and stirred for 16 hours at room temperature. Saturated brine was added to the reaction mixture, and the insoluble materials were removed by filtration through celite. The filtrate was diluted with water and extracted with ethyl acetate. The organic layer was washed with saturated brine and dried over anhydrous sodium sulfate. After the solvent was removed, the resulting residue was purified by column chromatography (silica gel, petroleum ether/ethyl acetate) to afford the title compound (9 g).

¹H NMR (DMSO-d₆) δ=7.93-7.84 (m, 1H), 6.89 (t, J=8.3 Hz, 1H), 3.93-3.80 (m, 1H), 2.62-2.59 (m, 2H), 1.36 (s, 9H), 1.12-1.07 (m, 12H). LC-MS (m/z): 291.20 [M+H]⁺.

### Second Step

### Production of tert-butyl {(2R,4RS)-4-[(tert-butylsulfinyl)amino]hept-5-yn-2-yl}carbamate

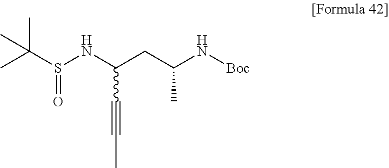 [SMILES: CC#CC(C[C@@H](C)NC(=O)OC(C)(C)C)NS(=O)C(C)(C)C]

To a solution of tert-butyl {(2R,E)-4-[(tert-butylsulfinyl)imino]butan-2-yl}carbamate (0.77 g, 2.65 mmol) in THE (10 mL), 1-propynylmagnesium bromide in 0.5 M-THF solution (21.2 mL, 10.62 mmol) was added at −20° C. and stirred for 16 hours at room temperature. To the reaction mixture, saturated aqueous ammonium chloride solution was added to quench the reaction, and the mixture was extracted with ethyl acetate. The organic layer was washed with saturated brine and dried over anhydrous sodium sulfate. After the solvent was removed, the resulting residue was purified by column chromatography (silica gel, petroleum ether/ethyl acetate) to afford the title compound (0.54 g).

¹H NMR (DMSO-d₆) δ=6.73-6.65 (m, 1H), 5.53-5.31 (m, 1H), 3.93-3.76 (m, 1H), 3.66-3.61 (m, 1H), 1.84-1.56 (m, 5H), 1.38 (s, 9H), 1.13-1.06 (m, 12H).

### Third Step

### Production of tert-butyl [(2R,4RS)-4-aminohept-5-yn-2-yl]carbamate

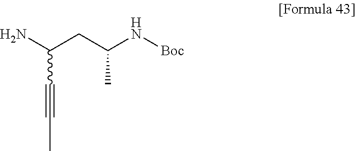 [SMILES: CC#CC(N)C[C@@H](C)NC(=O)OC(C)(C)C]

To a solution of tert-butyl {(2R,4RS)-4-[(tert-butylsulfinyl)amino]hept-5-yn-2-yl}carbamate (6 g, 18.18 mmol) in 1,4-dioxane (40 mL), 4 M hydrogen chloride/1,4-dioxane solution (4.54 mL, 181.8 mmol) was added at 0° C. and stirred for 16 hours at room temperature. Saturated sodium bicarbonate solution was added to the reaction mixture, the mixture was extracted with ethyl acetate, and the organic layer was washed with water and saturated brine successively and then dried over anhydrous sodium sulfate. After the solvent was concentrated under reduced pressure, the resulting residue was purified by column chromatography (silica gel, DCM/methanol) to afford the title compound (3.5 g).

¹H NMR (500 MHZ, DMSO-d₆) δ=6.79-6.65 (m, 1H), 3.66-3.56 (m, 1H), 3.43-3.41 (m, 1H), 1.77 (d, J=2.1 Hz, 3H), 1.65-1.55 (m, 1H), 1.50-1.42 (m, 1H), 1.37 (s, 9H), 1.01 (t, J=6.3 Hz, 3H).

### Fourth Step

### Production of tert-butyl {(2R,4RS)-4-[3-(2,3-dihydrobenzofuran-4-yl)thioureido]hept-5-yn-2-yl}carbamate

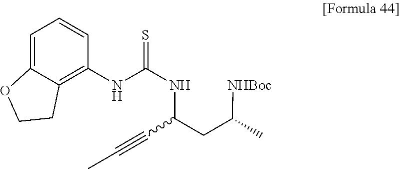 [SMILES: CC#CC(C[C@@H](C)NC(=O)OC(C)(C)C)NC(=S)Nc1cccc2c1CCO2]

To a solution of tert-butyl ((2R,4RS)-4-aminohept-5-yn-2-yl]carbamate (3.5 g, 15.49 mmol) in THE (30 mL), TEA (6.51 mL, 46.46 mmol) and 4-isothiocyanate-2,3-dihydrobenzofuran (Reference Example 1) (2.74 g, 15.49 mmol) were added and stirred for 16 hours at room temperature. After completion of the reaction, the reaction mixture was diluted with water and extracted with ethyl acetate. The organic layer was washed with saturated brine and dried over anhydrous sodium sulfate. After the solvent was removed, the resulting residue was purified by column chromatography (silica gel, petroleum ether/ethyl acetate) to afford the title compound (4 g). LC-MS (m/z): 404.21 [M+H]⁺.

### Fifth Step

### Production of tert-butyl {(2R,4RS)-4-(7,8-dihydrobenzofuro[4,5-d]thiazol-2-yl)amino]hept-5-yn-2-yl}carbamate

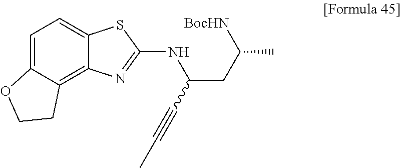 [SMILES: CC#CC(C[C@@H](C)NC(=O)OC(C)(C)C)Nc1nc2c3c(ccc2s1)OCC3]

To a solution of tert-butyl {(2R,4RS)-4-[3-(2,3-dihydrobenzofuran-4-yl)thioureido]hept-5-yn-2-yl}carbamate (4 g, 9.92 mmol) in chloroform (40 mL), sodium hydrogencarbonate (8.33 g, 99.25 mmol) and benzyltrimethylammonium tribromide (3.48 g, 8.93 mmol) were added at 0° C. and stirred for 3 hours. Water was added to the reaction mixture to quench the reaction, and the mixture was extracted with DCM. After the organic layer was washed with saturated brine and then dried over anhydrous sodium sulfate, the solvent was removed, and the resulting residue was purified by column chromatography (silica gel, petroleum ether/ethyl acetate) to afford the title compound (2.1 g).

LC-MS (m/z): 402.33 [M+H]⁺.

### Sixth Step

### Production of (2RS,4RS)—N4-(7,8-dihydrobenzofuro[4,5-d]thiazol-2-yl) hept-5-yn-2,4-diamine

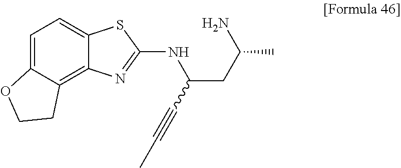 [SMILES: CC#CC(C[C@@H](C)N)Nc1nc2c3c(ccc2s1)OCC3]

To tert-butyl {(2R,4RS)-4-[(7,8-dihydrobenzofuro[4,5-d]thiazol-2-yl)amino]hept-5-yn-2-yl}carbamate (2.1 g, 5.24 mmol) in 1,4-dioxane (10 mL), 4 M hydrogen chloride/1,4-dioxane solution (7 mL) was added at 0° C. and stirred for 3 hours at room temperature. Toluene was added to the residue obtained by removing the solvent, and the remaining water was removed by toluene azeotrope to afford the title compound (2.1 g) as a crude product.

LC-MS (m/z): 302.20 [M+H]⁺.

### Seventh Step

### Production of (4R,6R)-1-(7,8-dihydrobenzofuro[4,5-d]thiazol-2-yl)-4-methyl-6-(prop-1-yn-1-yl)tetrahydropyrimidin-2(1H)-one

To a solution of (2R,4SR)—N4-(7,8-dihydrobenzofuro[4,5-d]thiazol-2-yl) hept-5-yn-2,4-diamine (2.1 g, 6.98 mmol) in DMF (15 mL), TEA (2.93 mL, 20.93 mmol) and N,N′-disuccinimidyl carbonate (1.78 g, 6.98 mmol) were added at 0° C. and stirred for 16 hours at room temperature. After water was added to the reaction mixture, the precipitated solid was collected by filtration to afford a crude product of the diastereomeric mixture consisting of (4R,6R)-1-(7,8-dihydrobenzofuro[4,5-d]thiazol-2-yl)-4-methyl-6-(prop-1-yn-1-yl)tetrahydropyrimidin-2(1H)-one and (4R,6S)-1-(7,8-dihydrobenzofuro[4,5-d]thiazol-2-yl)-4-methyl-6-(prop-1-yn-1-yl)tetrahydropyrimidin-2(1H)-one. The resulting diastereomeric mixture was purified by HPLC preparative chromatography to afford the titled compound (299 mg) as a fraction eluted first (retention time: 1.81 minutes).

¹H NMR (DMSO-d₆) δ=7.82 (d, J=2.7 Hz, 1H), 7.57 (d, J=8.3 Hz, 1H), 6.75 (d, J=8.3 Hz, 1H), 5.82-5.75 (m, 1H), 4.62 (t, J=8.8 Hz, 2H), 3.71-3.62 (m, 1H), 3.38 (t, J=8.8 Hz, 2H), 2.41-2.31 (m, 1H), 2.08-2.00 (m, 1H), 1.75 (d, J=2.2 Hz, 3H), 1.43 (d, J=6.8 Hz, 3H).

LC-MS (m/z): 328.24 [M+H]⁺.

### Example 15

### Production of (4R,6S)-1-(7,8-dihydrobenzofuro[4,5-d]thiazol-2-yl)-4-methyl-6-(prop-1-yn-1-yl)tetrahydropyrimidin-2(1H)-one

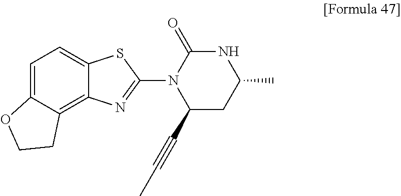 [SMILES: CC#C[C@@H]1C[C@@H](C)NC(=O)N1c1nc2c3c(ccc2s1)OCC3]

The diastereomeric mixture obtained in the seventh step of Example 14 was purified by HPLC preparative chromatography to afford the titled compound (29.5 mg) as a fraction eluted later (retention time: 1.85 minutes).

¹H NMR (DMSO-d₆) δ=7.74 (s, 1H), 7.57 (d, J=8.3 Hz, 1H), 6.75 (d, J=8.3 Hz, 1H), 5.84-5.77 (m, 1H), 4.62 (t, J=8.8 Hz, 2H), 3.91-3.83 (m, 1H), 3.43-3.36 (m, 2H), 2.19-2.12 (m, 1H), 1.85-1.77 (m, 4H), 1.21 (d, J=6.4 Hz, 3H). LC-MS (m/z): 328.24 [M+H]⁺.

TABLE 3

| Example No. | Structure | Compound Name |
|---|---|---|
| 16 | 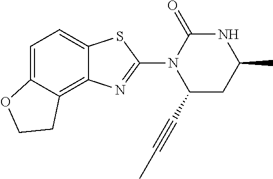 [SMILES: CC#C[C@H]1C[C@H](C)NC(=O)N1c1nc2c3c(ccc2s1)OCC3] | (4S,6S)-1-(7,8- dihydrobenzofuro[4,5- d]thiazol-2-yl)-4-methyl- 6-(prop-1-yn-1- yl)tetrahydropyrimidin- 2(1H)-one |
| 17 | 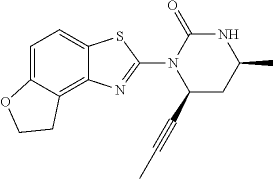 [SMILES: CC#C[C@@H]1C[C@H](C)NC(=O)N1c1nc2c3c(ccc2s1)OCC3] | (4S,6R)-1-(7,8- dihydrobenzofuro[4,5- d]thiazol-2-yl)-4-methyl- 6-(prop-1-yn-1- yl)tetrahydropyrimidin- 2(1H)-one |
| 18 (Enantiomer A of Ex. 2) | 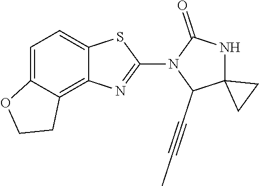 [SMILES: CC#CC1N(c2nc3c4c(ccc3s2)OCC4)C(=O)NC12CC2] | 6-(7,8- dihydrobenzofuro[4,5- d]thiazol-2-yl)-7-(prop-1- yn-1-yl)-4,6- diazaspiro[2.4]heptan-5- one |
| 19 (Enantiomer B of Ex. 2) | 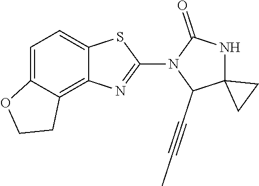 [SMILES: CC#CC1N(c2nc3c4c(ccc3s2)OCC4)C(=O)NC12CC2] | 6-(7,8- dihydrobenzofuro[4,5- d]thiazol-2-yl)-7-(prop-1- yn-1-yl)-4,6- diazaspiro[2.4]heptan-5- one |
| 20 (Enantiomer of Ex. 21) | 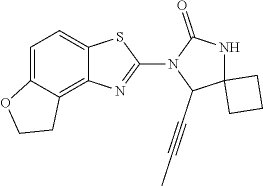 [SMILES: CC#CC1N(c2nc3c4c(ccc3s2)OCC4)C(=O)NC12CCC2] | 7-(7,8- dihydrobenzofuro[4,5- d]thiazol-2-yl)-8-(prop-1- yn-1-yl)-5,7- diazaspiro[3.4]octan-6-one |
| 21 (Enantiomer of Ex. 20) | 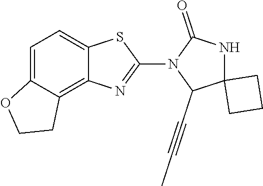 [SMILES: CC#CC1N(c2nc3c4c(ccc3s2)OCC4)C(=O)NC12CCC2] | 7-(7,8- dihydrobenzofuro[4,5- d]thiazol-2-yl)-8-(prop-1- yn-1-yl)-5,7- diazaspiro[3.4]octan-6-one |
| 22 (Enantiomer of Ex. 23) | 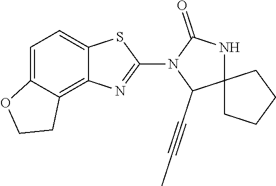 [SMILES: CC#CC1N(c2nc3c4c(ccc3s2)OCC4)C(=O)NC12CCCC2] | 3-(7,8- dihydrobenzofuro[4,5- d]thiazol-2-yl)-4-(prop-1- yn-1-yl)-1,3- diazaspiro[4.4]nonan-2-one |
| 23 (Enantiomer of Ex. 22) | 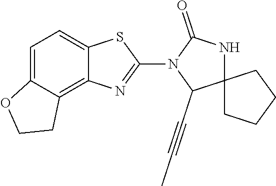 [SMILES: CC#CC1N(c2nc3c4c(ccc3s2)OCC4)C(=O)NC12CCCC2] | 3-(7,8- dihydrobenzofuro[4,5- d]thiazol-2-yl)-4-(prop-1- yn-1-yl)-1,3- diazaspiro[4.4]nonan-2-one |

TABLE 4

| | | LCMS m/z |
|---|---|---|
| Ex. No. | ¹H NMR δ (ppm) | [M + H] [symbol] |
| 16 | (DMSO-d6) δ = 7.74 ( [symbol] , 1H), 7.57 (d, J = 8.3 Hz, 1H), 6.7 [symbol] | 328.2 |
| | (d, J = 8.3 Hz, 1H), [symbol] .84-5.7 [symbol] (m, 1H), 4. [symbol] 2 ( [symbol] J = | |
| | 8.7 Hz, 2H), 3.91-3.80 (m, 1H), 3.43-3.35 (m, 2H), | |
| | 2.20-2.12 (m, 1H), 1.86-1.74 (m, 4H), 1.21 (d, J = 6.4 Hz, 3H) | |
| 17 | (DMSO-d6) δ 7.82 (br [symbol] s, 1H), 7.57 (d, J = 8.3 Hz, 1H), | 328.2 |
| | 6.75 (d, J = 8.6 Hz, 1H), 5. [symbol] 3-5.73 (m, 1H), 4.62 ( [symbol] | |
| | [symbol] J = 8.8 Hz, 2H), 3.71-3.61 (m, 1H), 3.38 ( [symbol] , J = 8 [symbol] 9 | |
| | Hz, 2H) [symbol] 2.41-2.31 (m, 1H) [symbol] 2.07-1.99 (m, 1H), 1.75 | |
| | (d, J = 2.2 Hz, 3H), 1.43 (d, J = 6.8 Hz, 3H) | |
| 18 | (DMSO-d6) δ 8.19 (br, s, 1H), 7.62 (d [symbol] J = 8.2 Hz, 1H) [symbol] | 326.2 |
| | 6.77 (d, J = 8. [symbol] Hz, 1H), 5.19 (d [symbol] J = 1.8 Hz, 1H), 4.65- | |
| | 4.59 (m, 2H), [symbol] -3.34 (m, 2H), 1.81 (d, J = 2.1 Hz, 3H), | |
| | 1.13-1. 06 (m, 1H), 1.0 [symbol] -0. [symbol] (m, 2H), 0.87-0.80 (m, 1H) [symbol] | |
| 19 | (DMSO-d6) δ 8.18 (s, 1H), 7.62 (d, J = 8.2 Hz, 1H), 6.77 | 326.2 |
| | (d, J = 8.5 Hz, 1H), [symbol] .19 (d, J = 2.1 Hz, 1H), 4.65-4.59 | |
| | (m, 2H), 3.36 ( [symbol] , J = 8.9 Hz, 2H), 1.81 (d, J = 1.8 Hz, 3H), | |
| | 1.14-1.06 (m, 1H), 0.99-0.90 (m, 2H), 0.87-0.80 (m, 1H) [symbol] | |
| 20 | (DMSO-d6) δ 8.53 (s, 1H), 7.61 (d, J = 8.3 Hz, 1H), 6.77 | 340.3 |
| | (d, J = 8.6 Hz, 1H), [symbol] .34 (d, J = 2.2 Hz, 1H), 4.63 (t, | |
| | J = [symbol] .0 Hz, 2H), 3.39 (t, J = 8.8 Hz, 2H), 2. [symbol] 8-2.53 | |
| | (m, 1H), 2.34-2.23 (m, 2H), 2.17-2.07 (m, 1H), 1.82 | |
| | (d, J = 2.0 Hz, 3H), 1.76-1.69 (m, 2H) [symbol] | |
| 21 | (DMSO-d6) δ 8. [symbol] 3 (s, 1H), 7.61 (d, J = [symbol] .3 Hz, 1H), 6.77 | 340.3 |
| | (d, J = 8.6 Hz, 1H), 5.34 (d, J = 2.2 Hz, 1H), 4.63 ( [symbol] , | |
| | J = 8.9 Hz, 2H), 3.39 (t, J = [symbol] .9 Hz, 2H), 2.59-2. [symbol] 3 | |
| | (m, 1H), 2.34-2.23 (m, 2H), 2.16-2.08 (m, 1H), 1.82 | |
| | (d, J = 2.0 Hz, 3H), 1.77-1.68 (m, 2H) | |
| 22 | (DMSO-d6) δ 8.30 (s, 1H), 7.61 (d, J = 8.3 Hz, 1H), 6.76 | 354.1 |
| | (d, J = [symbol] .3 Hz, 1H), 5.06 (d, J = 2.2 Hz, 1H), 4.62 ( [symbol] | |
| | J = 8.9 Hz, 2H), 3.41-3.35 (m, 2H), 3.12-3.01 (m, | |
| | 1H), 2.19-2.11 (m, 1H), 1.90-1.61 (m, [symbol] H) [symbol] | |
| 23 | (DMSO-d6) δ 8.30 (s, 1H), 7.61 (d, J = 8.4 Hz, 1H), 6.76 | 354.1 |
| | (d, J = 8.3 Hz, 1H), 5.06 (d, J = 2.0 Hz, 1H), 4.66- | |
| | [symbol] .58 (m, 2H), 3.41-3.34 (m, 2H), 3.0 [symbol] -3.05 (m, 1H) [symbol] | |
| | 2.21-2.11 (m, 1H), 1.90-1.62 (m, [symbol] H) | |

[symbol]

indicates data missing or illegible when filed

### Test Example 1

### Inhibition Test on Activity of DYRK Family (DYRK1A, DYRK1B, DYRK2, and DYRK3)

### (Method for Measuring Kinase Activity)

The kinase activity was measured by mobility shift assay (MSA) method using QuickScout Screening Assist™ MSA (commercially available kit manufactured by Carna Biosciences, Inc.). The substrate of the kinase reaction used was an FITC-labeled DYRKtide peptide included in the kit. An assay buffer [20 mM HEPES, 0.01% Triton X-100™, 2 mM dithiothreitol, pH 7.5] was used to create a substrate mixture solution with a substrate (4 μM), MgCl₂ (20 mM), and ATP (DYRK1A: 100 μM; DYRKIB: 200 μM; DYRK2:40 μM; and DYRK3:20 μM). In addition, kinases (DYRK1A: manufactured by Carna Biosciences, Inc., Cat. No. 04-130; DYRK1B: manufactured by Carna Biosciences, Inc., Cat. No. 04-131; DYRK2; manufactured by Carna Biosciences, Inc., Cat. No. 04-132; and DYRK3; manufactured by Carna Biosciences, Inc., Cat. No. 04-133) were diluted with the assay buffer to prepare enzyme solutions (DYRK1A: 0.2 ng/μL; DYRK1B: 0.08 ng/μL; DYRK2: 0.04 ng/μL; and DYRK3:0.25 ng/μL). The 10 mM solution of the test compound in DMSO was further diluted with DMSO to 10 levels of the concentration (0.00003 mM, 0.0001 mM, 0.0003 mM, 0.001 mM, 0.003 mM, 0.01 mM, 0.03 mM, 0.1 mM, 0.3 mM, and 1 mM), each of which was subjected to 25-fold dilution with the assay buffer to obtain a drug solution (4% DMSO solution). 5 μL of the drug solution or a control solution (4% DMSO-assay buffer), 5 μL of the substrate mixture solution, and 10 μL of the enzyme solution were mixed in the wells of a polypropylene 384-well plate and allowed to react at room temperature for 1 hour, and then the reaction was quenched by adding 60 μL of the termination buffer included in the kit. Subsequently, the quantities of the substrates(S) and the phosphorylated substrate (P) in the reaction solution were measured using LabChip EZ Reader II system (manufactured by Caliper Life Sciences) according to the protocol of the assay kit.

### (Method for Evaluating Inhibitory Activity)

The heights of the peaks of the “substrate” and the “phosphorylated substrate” were expressed as S and P, respectively, and a blank containing the assay buffer instead of the enzyme solution was also measured.

The inhibition rate (%) of the test compound was calculated according to the following equation:

$$Inhibition rate ( \% ) = ( 1 - ( C - A ) / ( B - A ) ) \times 100$$

wherein, A, B, and C represent P/(P+S) of the blank well, P/(P+S) of the control solution well, and P/(P+S) of the compound-containing well, respectively.

The IC₅₀ value was calculated via a regression analysis of the inhibition rate and the test compound concentration (logarithmic value).

### (Evaluation Result)

The inhibiting activities of representative compounds of the present invention are shown against DYRK1A, DYRK1B, DYRK2, and DYRK3 in [Table 5] and [Table 6]. The kinase activity inhibitory effect was indicated with the mark *** at an IC₅₀ value of less than 0.01 μM; the mark ** at 0.01 μM or more and less than 0.1 μM; the mark * at 0.1 μM or more and less than 1 μM; and the mark - at 1 μM or more (N.D. indicates not measured).

TABLE 5

| Test Compound | Inhibitory Activity | | | |
|---|---|---|---|---|
| Example No. | DYRK1A | DYRK1B | DYRK2 | DYRK3 |
| 1 | *** | N.D. | — | — |
| 2 | *** | N.D. | * | * |
| 3 | *** | N.D. | * | * |
| 4 | *** | N.D. | * | * |
| 5 | *** | *** | * | * |
| 6 | * | N.D. | — | — |
| 7 | *** | *** | * | * |
| 8 | ** | N.D. | — | — |
| 9 | ** | N.D. | — | * |

TABLE 6

| Test Compound | Inhibitory Activity | | | |
|---|---|---|---|---|
| Example No. | DYRK1A | DYRK1B | DYRK2 | DYRK3 |
| 10 | *** | N.D. | — | — |
| 11 | ** | N.D. | — | — |
| 12 | * | * | — | — |
| 13 | — | — | — | — |
| 14 | — | — | — | — |
| 15 | ** | ** | — | — |
| 16 | — | — | — | — |
| 17 | ** | ** | — | — |
| 18 | *** | N.D. | * | ** |
| 19 | — | N.D. | — | — |
| 20 | *** | N.D. | — | * |
| 21 | * | N.D. | — | — |
| 22 | *** | N.D. | — | * |
| 23 | * | N.D. | — | — |

These results have shown that the compounds (I) of the present invention have potent DYRK-inhibitory activities.

### INDUSTRIAL APPLICABILITY

The compound provided by the present invention is useful as a prophylactic or therapeutic agent for disease which is known to be involved in abnormal cell response through DYRK1A, for example, Alzheimer's disease, Parkinson's disease, Down's syndrome, mental retardation, memory impairment, memory loss, neuropsychiatric disorder such as depression, and cancers such as brain tumors. The compound is a DYRK1B inhibitor also useful as a prophylactic or therapeutic pharmaceutical (pharmaceutical composition) for cancers such as pancreatic cancer. Since DYRK2 controls p53 to induce apoptosis in response to DNA damages, the compound provided by the present invention is further useful as a prophylactic or therapeutic pharmaceutical (pharmaceutical composition) for bone resorption disease and osteoporosis. The compound provided by the present invention is a DYRK3 inhibitor also useful as a prophylactic or therapeutic pharmaceutical (pharmaceutical composition) for sickle-cell anemia, chronic kidney disease, bone resorption disease and osteoporosis. The compound is also useful, as a compound inhibiting DYRK, for reagents to be used in pathological imaging and for reagents for basic experiments and research regarding the above diseases.

1. A benzothiazole derivative of the following formula (I): 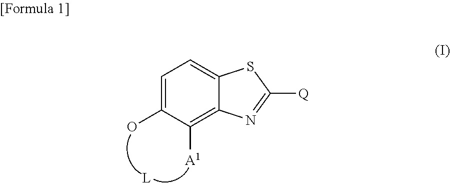
wherein A¹ represents an oxygen atom or optionally substituted methylene; L represents optionally substituted methylene or optionally substituted ethylene; and
Q represents the following structure (a) or (b): 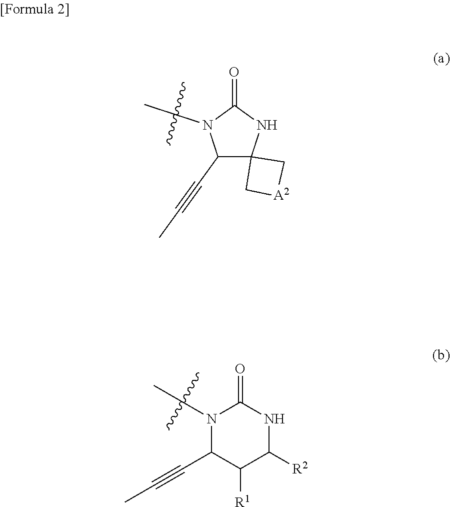
wherein A² represents, independently of A¹, a bond, optionally substituted methylene, optionally substituted ethylene, or an oxygen atom; and
R¹ and R² represent each independently a hydrogen atom or an optionally substituted lower alkyl group,
or a pharmaceutically acceptable salt thereof.

2. The benzothiazole derivative according to claim 1 above or a pharmaceutically acceptable salt thereof, wherein in formula (I) above, A¹ and L are methylene, and Q is structure (a) or (b).

3. The benzothiazole derivative according to claim 1 above or a pharmaceutically acceptable salt thereof, wherein in formula (I) above, A¹ is an oxygen atom, L is ethylene, and Q is structure (a) or (b).

4. The benzothiazole derivative according to claim 2 above or a pharmaceutically acceptable salt thereof, wherein Q is structure (a) in formula (I) above.

5. The benzothiazole derivative according to claim 2 above or a pharmaceutically acceptable salt thereof, wherein Q is structure (b) in formula (I) above.

6. The benzothiazole derivative according to claim 3 above or a pharmaceutically acceptable salt thereof, wherein Q is structure (a) in formula (I) above.

7. The benzothiazole derivative according to claim 3 above or a pharmaceutically acceptable salt thereof, wherein Q is structure (b) in formula (I) above.

8. A benzothiazole derivative according to Examples 1 to 23 or a pharmaceutically acceptable salt thereof.

9. A medicament comprising the benzothiazole derivative according to claim 1 above or a pharmaceutically acceptable salt thereof as an active ingredient.

10. The pharmaceutical composition comprising the benzothiazole derivative according to claim 1 above or a pharmaceutically acceptable salt thereof as an active ingredient.

11. A therapeutic agent and/or a prophylactic agent for a disease involving DYRK, comprising the benzothiazole derivative according to claim 1 above or a pharmaceutically acceptable salt thereof as an active ingredient.

12. The therapeutic agent and/or the prophylactic agent according to claim 11 above, wherein the disease involving DYRK is frontotemporal dementia, progressive supranuclear palsy, corticobasal degeneration, Lewy body dementia, vascular dementia, a traumatic brain injury, chronic traumatic encephalopathy, stroke, Alzheimer's disease, Parkinson's disease, Down's syndrome, depression and mental retardation associated therewith, memory impairment, memory loss, learning disability, intellectual disability, cognitive impairment, mild cognitive impairment, dementia symptoms or brain tumor, pancreatic cancer, ovarian cancer, osteosarcoma, colorectal cancer, lung cancer, bone resorption disease, osteoporosis, sickle cell anemia, chronic kidney disease or bone resorption disease.

13. A method for treating and/or preventing a disease involving DYRK, comprising administering to a patient in need of the treatment a therapeutically effective amount of a compound according to claim 1 above or a pharmaceutically acceptable salt thereof.

14. Use of the benzothiazole derivative according to claim 1 above or a pharmaceutically acceptable salt thereof, for producing a therapeutic agent and/or a prophylactic agent for a disease involving DYRK.

15. The benzothiazole derivative according to claim 1 above or a pharmaceutically acceptable salt thereof, for use in the treatment and/or prevention of a disease involving DYRK.

16. A medicament comprising the medicament according to claim 9 above in combination with at least one or more drugs selected from drugs classified as anticancer agents, antipsychotic drugs, antidementia drugs, antiepileptic drugs, antidepressants, gastrointestinal agents, thyroid hormone preparations, or antithyroid drugs.

17. The medicament according to claim 16 above, for treating frontotemporal dementia, progressive supranuclear palsy, corticobasal degeneration, Lewy body dementia, vascular dementia, a traumatic brain injury, chronic traumatic encephalopathy, stroke, Alzheimer's disease, Parkinson's disease, Down's syndrome, depression and a complication associated therewith, mental retardation, memory impairment, memory loss, learning disability, intellectual disability, cognitive impairment, mild cognitive impairment, dementia symptoms or brain tumor, pancreatic cancer, ovarian cancer, osteosarcoma, colorectal cancer, lung cancer, bone resorption disease, osteoporosis, sickle cell anemia, chronic kidney disease or bone resorption disease in combination with at least one or more drugs selected from drugs classified as anticancer agents, antipsychotic drugs, antidementia drugs, antiepileptic drugs, antidepressants, gastrointestinal agents, thyroid hormone preparations, or antithyroid drugs.
